(19) United States
(12) Patent Application Publication
(10) Pub. No.: US 2022/0267849 A1
(43) Pub. Date: Aug. 25, 2022

(54) TRANSPLANT PATIENT MONITORING WITH CELL-FREE DNA

(71) Applicant: The Medical College of Wisconsin, Inc., Milwaukee, WI (US)

(72) Inventors: Aoy Tomita Mitchell, Elm Grove, WI (US); Michael Mitchell, Elm Grove, MI (US)

(21) Appl. No.: 17/493,293

(22) Filed: Oct. 4, 2021

Related U.S. Application Data
(63) Continuation of application No. PCT/US2020/026600, filed on Apr. 3, 2020.
(60) Provisional application No. 62/829,004, filed on Apr. 3, 2019.

(51) Int. Cl. C12Q 1/6881; C12Q 1/6883; C12Q 1/6858; C12Q 1/6827

(52) U.S. Cl. CPC C12Q 1/6881; C12Q 1/6883; C12Q 1/6858; C12Q 1/6827; C12Q 2600/118; C12Q 2600/156; C12Q 2600/16

(57) ABSTRACT

This invention relates to methods and compositions for assessing risk by obtaining the product of a donor-specific fraction and total cell-free DNA, such as from a transplant subject. The methods and compositions provided herein can be used to assess a transplant subject to determine risk of one or more transplant complications (e.g., death) and/or a need for monitoring or treatment.

### RELATED APPLICATIONS

This application claims the benefit of priority under 35 U.S.C. § 119(e) to U.S. Provisional Application, U.S. Ser. No. 62/829,004, filed Apr. 3, 2019, the contents of which are incorporated by reference herein in their entirety.

### FIELD OF THE INVENTION

This invention relates to methods and compositions for assessing risk by measuring an amount of total cell-free nucleic acids, an amount of donor-specific cell-free nucleic acids in samples and/or a product thereof, for example, from a transplant subject. Such amounts can be used to monitor and/or determine risk of a condition, such as a transplant rejection.

### SUMMARY OF INVENTION

It has been found that the product of an amount of total cell-free nucleic acids and an amount of donor-specific cell-free nucleic acids has increased power in assessing risk. The product can be used to monitor and/or evaluate a subject as provided herein and can be beneficial in assessing the subject and allow for any needed intervention. Thus, the methods and compositions provided herein can be used to monitor transplant subjects over time post transplant. Deviations from a “normal” or other threshold or cutpoint (cutoff) may be indicative of one or more transplant complications, such as death, and/or need for additional monitoring or treatment. Using any one of a variety of means to quantify the total cell-free DNA and donor-specific cell-free DNA in samples from a transplant subject (and/or product thereof), the risk of complications following transplantation can be determined as well as monitored over time.

Provided herein are methods, compositions and kits related to such a determination. The methods, compositions, or kits can be any one of the methods, compositions, or kits, respectively, provided herein, including any one of those of the Examples or Figures.

In one embodiment of any one of the methods provided, the method further comprises obtaining a sample from the subject.

In one embodiment, any one of the embodiments for the methods provided herein can be an embodiment for any one of the compositions, kits or reports provided. In one embodiment, any one of the embodiments for the compositions, kits or reports provided herein can be an embodiment for any one of the methods provided herein.

In one aspect, a report or database comprising one or more of the amounts provided herein is provided.

In one aspect, a method of treating a subject, determining a treatment regimen for a subject or providing information about a treatment to the subject, based on the product or any one of the methods of analysis provided herein is provided. In one embodiment of any one of such methods, the method comprises a step of treating the subject or providing information about a treatment to the subject. In one embodiment of any one of the methods of treating, the treatment may be any one of the treatments provided herein. In one embodiment of any one of the methods of treating, the treatment is for any one of the conditions provided herein. Examples of which are provided herein or otherwise known to those of ordinary skill in the art.

In one aspect, any one of the methods provided herein may be a method of treating a transplant subject, such as a cardiac transplant subject, such as a pediatric cardiac transplant subject or adult cardiac transplant subject.

In one embodiment, the product value is determined in any way provided herein including in the Examples or Figures.

### BRIEF DESCRIPTION OF FIGURES

The accompanying figures are not intended to be drawn to scale. The figures are illustrative only and are not required for enablement of the disclosure.

FIG. 1 provides an exemplary, non-limiting diagram of MOMA primers. In a polymerase chain reaction (PCR) assay, extension of the sequence containing SNV A is expected to occur, resulting in the detection of SNV A, which may be subsequently quantified. Extension of the SNV B, however, is not expected to occur due to the double mismatch.

FIG. 2 shows results from a reconstruction experiment.

FIG. 3 demonstrates the use of expectation maximization to predict donor genotype. Dashed line=first iteration, Solid line=second iteration, Final call=10%.

FIG. 4 demonstrates the use of expectation maximization to predict donor genotype. Final call=5%. Shows results from a reconstruction experiment.

FIG. 5 provides reconstruction experiment data demonstrating the ability to predict the donor genotype. Data were generated with a set of 95 SNV targets.

FIG. 6 illustrates an example of a computer system with which some embodiments may operate.

FIGS. 7A-7D show an experimental determination of the correlation between total cf-DNA (whole blood and plasma) and death in all subjects.

FIGS. 8A-8C show an experimental determination of the correlation between total cf-DNA (whole blood and plasma) and death using the last sample from each subject.

FIGS. 9A-9B show an experimental determination of the correlation between total cf-DNA (whole blood and plasma) and death in pediatric subjects.

FIGS. 10A-10B show an experimental determination of the correlation between total cf-DNA (whole blood and plasma) and death in adult subjects.

FIGS. 11A-11D show an experimental determination of the correlation between donor-specific cf-DNA (whole blood and plasma) and death in all subjects.

FIGS. 12A-12C show an experimental determination of the cutpoint and correlation between donor-specific cf-DNA (whole blood and plasma) and death using the last sample from each subject.

FIGS. 13A-13B show an experimental determination of the cutpoint and correlation between donor-specific cf-DNA (whole blood and plasma) and death in pediatric subjects.

FIGS. 14A-14B show an experimental determination of the cutpoint and correlation between donor-specific cf-DNA (whole blood and plasma) and death in adult subjects.

FIGS. 15A-15B show the cutpoint and correlation of donor-specific cf-DNA and total cf-DNA with death, as determined by the last sample per subject.

FIGS. 16A-16B show the cutpoint and correlation of donor-specific cf-DNA and total cf-DNA with death, as determined by all samples.

FIGS. 17A-17B show an experimental determination of the cutpoint and correlation between total cf-DNA (whole blood and plasma) and graft vasculopathy in pediatric subjects.

FIGS. 18A-18B show an experimental determination of the cutpoint and correlation between donor-specific cf-DNA (whole blood and plasma) and graft vasculopathy in pediatric subjects (one sample per subject).

FIGS. 19A-19B show an experimental determination of the cutpoint and correlation between total cf-DNA (plasma) and rejection (e.g., acute cellular rejection (ACR) levels of 1, 2, or 3 and/or antibody-mediated rejection (AMR) levels of 1 or 2).

FIGS. 20A-20B show an experimental determination of the cutpoint and correlation between donor-specific cf-DNA (plasma) and rejection (e.g., acute cellular rejection (ACR) levels of 1, 2, or 3 and/or antibody-mediated rejection (AMR) levels of 1 or 2).

FIGS. 21A-21B show an experimental determination of the cutpoint and correlation between total cf-DNA (plasma) and rejection (e.g., acute cellular rejection (ACR) levels of 1, 2, or 3 and/or antibody-mediated rejection (AMR) levels of 1 or 2) in pediatric subjects.

FIGS. 22A-22B show an experimental determination of the cutpoint and correlation between donor-specific cf-DNA (plasma) and rejection (e.g., acute cellular rejection (ACR) levels of 1, 2, or 3 and/or antibody-mediated rejection (AMR) levels of 1 or 2) in pediatric subjects.

FIGS. 23A-23B show an experimental determination of the cutpoint and correlation between total cf-DNA (in whole blood and plasma) and treatment for infection.

FIGS. 24A-24B show an experimental determination of the cutpoint and correlation between donor-specific cf-DNA (in whole blood and plasma) and treatment for infection.

FIGS. 25A-25B show an experimental determination of the cutpoint and correlation between total cf-DNA (in whole blood and plasma) and treatment for infection using one sample per subject.

FIGS. 26A-26B show an experimental determination of the cutpoint and correlation between donor-specific cf-DNA (in whole blood and plasma) and treatment for infection using one sample per subject.

FIG. 27 depicts an exemplary classification of regression tree (CART) to assess risk.

FIG. 28 depicts a risk analysis using a linear discriminant analysis (LDA).

FIGS. 29A-29C show an analysis of the correlation of donor specific cf-DNA and total cf-DNA with death with respect to collection time (FIG. 29A), as well as donor specific cf-DNA alone (FIG. 29B) and total cf-DNA (FIG. 29C) alone.

FIGS. 30A-30C show an experimental determination of the correlation between donor-specific cf-DNA and total cf-DNA with death using all available samples.

FIGS. 31A-31C show an experimental determination of the correlation between donor-specific cf-DNA and total cf-DNA with death using the last sample per subject.

FIGS. 32A-32C show an analysis of different cutpoints with respect to total cf-DNA and death: 50 ng/mL (FIG. 32A), 25 ng/mL (FIG. 32B), and 10 ng/mL (FIG. 32C).

### DETAILED DESCRIPTION OF THE INVENTION

Aspects of the disclosure relate to methods for assessing risk (e.g., of death) in a subject. The risk can be assessed, in some embodiments, using a combination of one or more values for the amount of total cell-free nucleic acids (such as DNA; cf-DNA) and one or more values for the amount of donor-specific (donor fraction) cell-free nucleic acids (such as DNA) in a subject. In some embodiments, risk is assessed using the product of an amount of total cell-free nucleic acids (such as cfDNA) and an amount of donor-specific cell-free nucleic acids (such as DNA). In some embodiments of any one of the methods provided herein the product value can be calculated by multiplying an amount of donor-specific cell-free nucleic acids (such as DNA) and an amount of total cell-free nucleic acids (such as DNA). In some embodiments of any one of the methods provided herein the product value can be calculated by multiplying an amount of donor-specific cell-free nucleic acids (such as DNA) and an amount of total cell-free nucleic acids (such as DNA) and a conversion factor such as any of the conversion factors provided herein including in the Examples and Figures (e.g., 302 haploid per nanogram or reciprocal of 6.6 pg/diploid). Deviations from a “normal” or threshold or cutpoint or cutoff can be indicative of a transplant complication and/or need for additional monitoring or treatment. Determining and/or monitoring amounts of these nucleic acids, or the product thereof, is beneficial to assess a transplant subject and allow for any needed intervention. In any one of the methods provided herein, the threshold or cutpoint or cutoff may be any one of the thresholds or cutpoints or cutoffs herein. Therefore, aspects of the disclosure relate, at least in part, to methods of quantifying donor-specific cell-free DNA (DS cf-DNA) and total cf-DNA in a number of samples (or product thereof) from a subject in order to assess or determine the health of the subject and/or transplant.

As used herein, “donor-specific nucleic acids” or “donor fraction nucleic acids” refer to nucleic acids that are from a transplant donor that can be found in a transplant recipient. Such nucleic acids are preferably cell-free DNA. “Cell-free DNA” (or “cf-DNA”) is DNA that is present outside of a cell, e.g., in the blood, plasma, serum, urine, etc. of a subject. “Total cell-free DNA” (or “total cf-DNA”) is the amount of cf-DNA present in a sample, and can include both donor and recipient cf-DNA when assessing a sample from a transplant recipient. As used herein, the compositions and methods provided herein can be used to determine an amount of DS cf-DNA and total cell-free DNA (and/or product thereof) and a subject's risk of complications associated with a transplant. As used herein, “transplant” refers to an organ or tissue from one source, such as from a donor, to a recipient for the purpose of replacing or adding to the recipient's organ or tissue, such as a damaged or absent organ or tissue. Any one of the methods or compositions provided herein may be used on a sample from a subject that has undergone a transplant of an organ or tissue. In some embodiments, the transplant is a heart transplant.

A subject may be assessed by determining or obtaining one or more amounts of cf-DNA as described herein. An amount of cf-DNA may be determined with experimental techniques, such as those provided elsewhere herein. “Obtaining” as used herein refers to any method by which the respective information or materials can be acquired. Thus, the respective information can be acquired by experimental methods. Respective materials can be created, designed, etc. with various experimental or laboratory methods, in some embodiments. The respective information or materials can also be acquired by being given or provided with the information, such as in a report, or materials. Materials may be given or provided through commercial means (i.e. by purchasing), in some embodiments.

A “risk” as provided herein, refers to the presence or absence of any undesirable condition in a subject (such as a transplant recipient), or an increased likelihood of the presence or absence of such a condition, e.g., transplant rejection, infection. As provided herein, “increased risk” refers to the presence of any undesirable condition in a subject or an increased likelihood of the presence of such a condition. As provided herein, “decreased risk” refers to the absence of any undesirable condition in a subject or a decreased likelihood of the presence (or increased likelihood of the absence) of such a condition. As an example, early detection of rejection following implantation of a transplant (e.g., a heart transplant) can facilitate treatment and improve clinical outcomes. Transplant rejection remains a major cause of graft failure and late mortality and generally requires lifelong surveillance monitoring. Treatment of transplant rejections with immunosuppressive therapy has been shown to improve treatment outcomes, particularly if rejection is detected early.

Accordingly, in some embodiments of any one of the methods provided, the subject is a recipient of a transplant, and the risk is a risk associated with the transplant. In some embodiments of any one of the methods provided, the risk associated with the transplant is risk of transplant rejection, an anatomical problem with the transplant or injury to the transplant. In some embodiments of any one of the methods provided, the injury to the transplant is initial or ongoing injury. In some embodiments of any one of the methods provided, the risk associated with the transplant is an acute condition or a chronic condition. In some embodiments of any one of the methods provided, the risk associated with the transplant is indicative of the severity of the injury. In some embodiments of any one of the methods provided, the risk is indicative of the overall health of a transplant. However, in some embodiments, the foregoing may be based on one or more other tests, such as with a biopsy. In such an embodiment, the methods provided herein can be used to confirm such a finding or monitor such a subject for worsening or improving condition.

The amounts provided herein (and/or product thereof) can be used to assess or determine risk of death in any one of the subjects provided herein. In such an embodiment, the methods provided herein can be used to monitor a subject with increased risk for worsening or improving condition over time. Thus, any one of the methods provided herein can include monitoring the subject more frequently or treating or modifying a treatment for a subject.

As provided herein, any one of the methods can be used to assess a subject that has or is suspected of having a transplant complication. In one embodiment of any one of the methods provided herein, the subject may be one that has a transplant complication or that a clinician believes there is a likelihood of having a transplant complication. In some embodiments, any one of the methods can be used to assess a subject that has had or is at risk of having a transplant complication. Subjects may be suspected of having, determined to have had, or determined to have a likelihood or risk of having a transplant complication based on symptoms (and/or lack thereof). However, in some embodiments, the subject is suspected of having, determined to have had, or determined to have a likelihood or risk of having a transplant complication based on one or more other tests. In such an embodiment, the methods provided herein can be used to confirm such a finding or monitor such a subject for worsening or improving condition.

An amount of cf-DNA (DS and/or total) may be determined with experimental techniques, such as those provided elsewhere herein. “Obtaining” as used herein refers to any method by which the respective information or materials can be acquired. Thus, the respective information can be acquired by experimental methods. Respective materials can be created, designed, etc. with various experimental or laboratory methods, in some embodiments. The respective information or materials can also be acquired by being given or provided with the information, such as in a report, or materials. Materials may be given or provided through commercial means (i.e. by purchasing), in some embodiments.

As provided herein, early detection or monitoring can facilitate treatment and improve clinical outcomes. Any one of the methods provided can be performed on any one of the subjects provided herein. Such methods can be used to monitor a subject over time, with or without treatment. Further, such methods can aid in the selection, administration and/or monitoring of a treatment or therapy. Accordingly, the methods provided herein can be used to determine a treatment or monitoring regimen.

“Determining a treatment regimen”, as used herein, refers to the determination of a course of action for treatment of the subject. In one embodiment of any one of the methods provided herein, determining a treatment regimen includes determining an appropriate therapy or information regarding an appropriate therapy to provide to a subject. In some embodiments of any one of the methods provided herein, the determining includes providing an appropriate therapy or information regarding an appropriate therapy to a subject. As used herein, information regarding a treatment or therapy or monitoring may be provided in written form or electronic form. In some embodiments, the information may be provided as computer-readable instructions. In some embodiments, the information may be provided orally.

The therapies can be, for example, an anti-rejection therapy. Anti-rejection therapies include, for example, immunosuppressives. Immunosuppressives include, but are not limited to, corticosteroids (e.g., prednisolone or hydrocortisone), glucocorticoids, cytostatics, alkylating agents (e.g., nitrogen mustards (cyclophosphamide), nitrosoureas, platinum compounds, cyclophosphamide (Cytoxan)), antimetabolites (e.g., folic acid analogues, such as methotrexate, purine analogues, such as azathioprine and mercaptopurine, pyrimidine analogues, and protein synthesis inhibitors), cytotoxic antibiotics (e.g., dactinomycin, anthracyclines, mitomycin C, bleomycin, mithramycin), antibodies (e.g., anti-CD20, anti-IL-1, anti-IL-2Ralpha, anti-T-cell or anti-CD-3 monoclonals and polyclonals, such as Atgam, and Thymoglobuline), drugs acting on immunophilins, ciclosporin, tacrolimus, sirolimus, interferons, opiods, TNF-binding proteins, mycophenolate, fingolimod and myriocin. In some embodiments, anti-rejection therapy comprises blood transfer or marrow transplant. Therapies can also include intravenous fluids, antibiotics, surgical drainage, early goal directed therapy (EGDT), vasopressors, steroids, activated protein C, drotrecogin alfa (activated), oxygen and appropriate support for organ dysfunction. This may include hemodialysis in kidney failure, mechanical ventilation in pulmonary dysfunction, transfusion of blood products, and drug and fluid therapy for circulatory failure. Ensuring adequate nutrition—preferably by enteral feeding, but if necessary, by parenteral nutrition—can also be included particularly during prolonged illness. Other associated therapies can include insulin and medication to prevent deep vein thrombosis and gastric ulcers.

The therapies can also include, for example, immunosuppressives, plasmapheresis/plasma exchange, intravenous immunoglobulin, corticosteroids, anti-lymphocyte antibodies, and splenectomy. The therapies can also include, for example, retransplantation, percutaneous coronary interventions (PCI), coronary artery bypass grafting (CABG), transmyocardial laser revascularization and/or heparin-induced/mediated extracorporeal LDL plasmapheresis (HELP), as well as the administration of statins, anti-hypertensive agents, and/or anti-cytomegalovirus (anti-CMV) agents.

In another embodiment, the treatment can be a treatment for infection. In some embodiments, therapies for treating infection include therapies for treating a bacterial, fungal and/or viral infection. Such therapies include antibiotics. Other examples include, but are not limited to, amebicides, aminoglycosides, anthelmintics, antifungals, azole antifungals, echinocandins, polyenes, diarylquinolines, hydrazide derivatives, nicotinic acid derivatives, rifamycin derivatives, streptomyces derivatives, antiviral agents, chemokine receptor antagonist, integrase strand transfer inhibitor, neuraminidase inhibitors, NNRTIs, NS5A inhibitors, nucleoside reverse transcriptase inhibitors (NRTIs), protease inhibitors, purine nucleosides, carbapenems, cephalosporins, glycylcyclines, leprostatics, lincomycin derivatives, macrolide derivatives, ketolides, macrolides, oxazolidinone antibiotics, penicillins, beta-lactamase inhibitors, quinolones, sulfonamides, and tetracyclines.

Other such therapies are known to those of ordinary skill in the art.

Administration of a treatment or therapy may be accomplished by any method known in the art (see, e.g., Harrison's Principle of Internal Medicine, McGraw Hill Inc.). Preferably, administration of a treatment or therapy occurs in a therapeutically effective amount. Administration may be local or systemic. Administration may be parenteral (e.g., intravenous, subcutaneous, or intradermal) or oral. Compositions for different routes of administration are known in the art (see, e.g., Remington's Pharmaceutical Sciences by E. W. Martin).

In some aspects, the methods include steps for determining a value for the amount of total cell-free nucleic acids (such as DNA), a value for the amount of specific cell-free nucleic acids (such as DNA) and/or a product of such values. As used herein, a “value” is any indicator that conveys information about an “amount”. The indicator can be an absolute or relative value for the amount. Further, the value can be the amount, frequency, ratio, percentage, etc. As used herein, the term “level” can be used instead of “amount” but is intended to refer to the same types of values.

In some instances the values can be compared to a “threshold value”. As used herein, a “threshold value” refers to any predetermined level or range of levels that is indicative of a state, the presence or absence of a condition or the presence or absence of a risk. The threshold value can take a variety of forms. It can be single cut-off value, such as a median or mean. It can be established based upon comparative groups, such as where the risk in one defined group is double the risk in another defined group. It can be a range, for example, where the tested population is divided equally (or unequally) into groups, such as a low-risk group, a medium-risk group and a high-risk group, or into quadrants, the lowest quadrant being subjects with the lowest risk and the highest quadrant being subjects with the highest risk. For example, an apparently healthy population will have a different ‘normal’ range. As another example, a threshold value can be determined from baseline values before the presence of a state, condition or risk or after a course of treatment. Such a baseline can be indicative of a normal or other state in the subject not correlated with the risk or condition that is being tested for. In some embodiments, the threshold value can be a baseline value of the subject being tested. Accordingly, the predetermined values selected may take into account the category in which the subject falls. Appropriate ranges and categories can be selected with no more than routine experimentation by those of ordinary skill in the art. The threshold value of any one of the methods, reports, databases, etc. provided herein, can be any one of the threshold values provided herein, such as in the Examples or Figures.

The treatment and clinical course may be determined by the subject's assessed risk. For example, if the product of the amount of donor fraction cf-DNA and total cf-DNA is equal to or greater than any one of the thresholds provided herein, an increased risk may be determined, and the subject may be treated with, or provided information related thereto, anti-rejection therapies, such as those described above. In one embodiment of any one of such methods, the subject may be treated with, or provided information related thereof, a different or more or less of a specific therapeutic intervention.

“Determining a monitoring regimen”, as used herein, refers to determining a course of action to monitor a condition in the subject over time. In one embodiment of any one of the methods provided herein, determining a monitoring regimen includes determining an appropriate course of action for determining the amount of DS cf-DNA, total cf-DNA in the subject and/or a product thereof over time or at a subsequent point in time, or suggesting such monitoring to the subject. This can allow for the measurement of variations in a clinical state and/or permit calculation of normal values or baseline levels (as well as comparisons thereto).

In some embodiments of any one of the methods provided herein determining a monitoring regimen includes determining the timing and/or frequency of obtaining samples from the subject and/or determining or obtaining an amount of DS cf-DNA and total cf-DNA (and/or product thereof).

In some embodiments, amounts of DS cf-DNA and total cf-DNA (and/or product thereof) can be plotted over time. In some embodiments, threshold values for the points in time may also be plotted. Such threshold values can be determined using data from a sufficient number of subjects. A comparison with a subject's levels to such threshold values over a period of time can be used to predict risk.

As increasing levels have been found to correlate with an increased risk of transplant complications, a clinician may determine that a subject should undergo more frequent sampling if the subject's levels are found to increase between time points. If a subject is found to have decreasing levels between time points, a clinician may determine that less frequent sampling is sufficient. Timing and/or frequency of monitoring may also be determined by a comparison to threshold values.

In some embodiments of any one of the methods provided herein, each amount and time point may be recorded in a report or in a database. Threshold values may also be recorded in a report or in a database.

Reports with any one or more of the values as provided herein are also provided in an aspect. Reports may be in oral, written (or hard copy) or electronic form, such as in a form that can be visualized or displayed. Preferably, the report provides the amount of donor-specific and total nucleic acids (or product thereof) in a sample. In some embodiments, the report provides amounts of donor-specific nucleic acids and total nucleic acids (and/or product thereof) in samples from a subject over time, and can further include corresponding threshold values in some embodiments.

In some embodiments, the amounts and/or values are in or entered into a database. In one aspect, a database with such amounts and/or values is provided. From the amount(s) and/or value(s), a clinician may assess the need for a treatment or monitoring of a subject. Accordingly, in any one of the methods provided herein, the method can include assessing the amounts and/or values in the subject at more than one point in time. Such assessing can be performed with any one of the methods or compositions provided herein.

In some embodiments, any one of the methods provided herein can comprise comparing an amount of donor-specific nucleic acids and total nucleic acids, or a product of the donor-specific cf-DNA and total cf-DNA, to a threshold value, or to one or more prior product values, to identify a subject at increased or decreased risk. In some embodiments of any one of the methods provided herein, a subject having an increased value compared to a threshold value, or to one or more prior values, is identified as being at increased risk. In some embodiments of any one of the methods provided herein, a subject having a decreased or similar value of compared to a threshold value, or to one or more prior values, is identified as being at decreased or not increased risk.

Any one of the methods provided herein may further include an additional test(s) for assessing the subject, or a step of suggesting such further testing to the subject (or providing information about such further testing). The additional test(s) may be any one of the methods provided herein. The additional test(s) may be any one of the other methods provided herein or otherwise known in the art as appropriate.

Exemplary additional tests for subjects, include, but are not limited to, echocardiogram, coronary angiography, intravascular ultrasound (IVUS), biopsy (e.g., endomycardial biopsy), stress echocardiography, CT coronary angiography, coronary flow reserve assessment (contrast-enhanced echocardiography), stress myocardial perfusion scintigraphy, positron emission tomography (PET) scanning, and measurement of serum biomarkers, such as BNP and/or troponin. In other embodiments of any one of the methods provided herein, the other test in addition to the level of BNP and/or troponin or in place thereof, is an echocardiogram.

Exemplary additional tests include, but are not limited to, presence of donor-specific antibody (HLA antibodies), positive C4d staining on biopsy (e.g., renal biopsy, endomycardial biopsy), and histopathological evidence of antibody-mediated injury (e.g., glomerulitis, peritubular capillaritis, arteritis).

Other examples of additional tests, include, but are not limited to, such as for subjects suspected of infection include, but are not limited to, blood tests, urine tests, throat swabs, and spinal tap.

The type of additional test(s) will depend upon the severity of the subject's condition and/or is well within the determination of the skilled artisan.

The amount of cf-DNA, DS and/or total, or product thereof, may be determined by a number of methods. Methods for determining total cell-free nucleic acids (such as DNA) as well as specific cell-free nucleic acids (such as DNA) are provided herein or are otherwise known in the art. For example, the methods of PCT Application No. PCT/US2016/030313 may be used for determining a value for the amount of specific cell-free nucleic acids (such as DNA) in a sample as provided herein. Thus, any one of the methods provided herein may include the steps of any one of the methods described in PCT Application No. PCT/US2016/030313, and such methods and steps are incorporated herein by reference. Likewise, the methods of measuring cell-free DNA of U.S. Publication No. US-2015-0086477-A1 are also incorporated herein by reference and such methods can be included as part of any one of the methods provided herein for determining a value for the amount of specific cell-free nucleic acids (such as DNA).

As a further example, real-time PCR may be used to determine a value for the amount of total cell-free nucleic acids (such as DNA). For example, in some embodiments of any one of the methods provided herein, the total cell-free nucleic acids (such as DNA) is determined with Taqman Real-time PCR using RNase P as a target. Other methods would be apparent to those of ordinary skill in the art.

In some embodiments such a method is a sequencing-based method. For example, the cf-DNA may be measured by analyzing the DNA of a sample to identify multiple loci, an allele of each of the loci may be determined, and informative loci may be selected based on the determined alleles. As used herein, “loci” refer to nucleotide positions in a nucleic acid, e.g., a nucleotide position on a chromosome or in a gene. As used herein, “informative loci” refers to a locus where the genotype of the subject is homozygous for the major allele, while the genotype of the donor is homozygous or heterozygous for the minor allele. As used herein, “minor allele” refers to the allele that is less frequent in the population of nucleic acids for a locus. In some embodiments, the minor allele is the nucleotide identity at the locus in the nucleic acid of the donor. A “major allele”, on the other hand, refers to the more frequent allele in a population. In some embodiments, the major allele is the nucleotide identity at the locus in the nucleic acid of the subject.

In some embodiments, the informative loci and alleles can be determined based on prior genotyping of the nucleic acids of the subject and the nucleic acids of the donor. For example, the genotype of the recipient and donor can be compared, and informative loci can be identified as those loci where the recipient is homozygous for a nucleotide identity and the donor is heterozygous or homozygous for a different nucleotide identity. Methods for genotyping are well known in the art and further described herein. In this example, the minor and major allele may be identified by determining the relative quantities of each allele at the informative locus and/or may be identified as the nucleotide identity at the informative locus in the donor DNA (minor allele) and the recipient DNA (major allele). Accordingly, the methods provided can further include a step of genotyping the recipient and donor, or obtaining or being provided with such genotypes.

An estimated allele frequency, such as the estimated minor allele frequency, at the informative loci may then be calculated in a suitable manner. In some embodiments, the estimated allele frequency may be calculated based on modeling the number of counts of the allele, such as the minor allele, at the informative loci using a statistical distribution. For example, the estimated allele frequency can be calculated by modeling allele read counts using a binomial distribution. In some embodiments, the peak of such a distribution is determined and is indicative of the percent donor-specific cf-DNA. A frequency of the minor allele at the informative loci may also be calculated using a maximum likelihood method. In some embodiments, the minor allele frequency (MAF) may be calculated with genotypes from plasma DNA of the subject, and donor genotypes for informative loci may be inferred using expectation maximization. In some embodiments, the read counts for the major and/or minor allele(s) can be corrected prior to estimating the allele frequency.

The DNA may be analyzed using any suitable next generation or high-throughput sequencing and/or genotyping technique. Examples of next generation and high-throughput sequencing and/or genotyping techniques include, but are not limited to, massively parallel signature sequencing, polony sequencing, 454 pyrosequencing, Illumina (Solexa) sequencing, SOLiD sequencing, ion semiconductor sequencing, DNA nanoball sequencing, heliscope single molecule sequencing, single molecule real time (SMRT) sequencing, MassARRAY®, and Digital Analysis of Selected Regions (DANSR™) (see, e.g., Stein R A (1 Sep. 2008). “Next-Generation Sequencing Update”. Genetic Engineering & Biotechnology News 28 (15); Quail, Michael; Smith, Miriam E; Coupland, Paul; Otto, Thomas D; Harris, Simon R; Connor, Thomas R; Bertoni, Anna; Swerdlow, Harold P; Gu, Yong (1 Jan. 2012). “A tale of three next generation sequencing platforms: comparison of Ion torrent, pacific biosciences and illumina MiSeq sequencers”. BMC Genomics 13 (1): 341; Liu, Lin; Li, Yinhu; Li, Siliang; Hu, Ni; He, Yimin; Pong, Ray; Lin, Danni; Lu, Lihua; Law, Maggie (1 Jan. 2012). “Comparison of Next-Generation Sequencing Systems”. Journal of Biomedicine and Biotechnology 2012: 1-11; Qualitative and quantitative genotyping using single base primer extension coupled with matrix-assisted laser desorption/ionization time-of-flight mass spectrometry (MassARRAY®). Methods Mol Biol. 2009; 578:307-43; Chu T, Bunce K, Hogge W A, Peters D G. A novel approach toward the challenge of accurately quantifying fetal DNA in maternal plasma. Prenat Diagn 2010; 30:1226-9; and Suzuki N, Kamataki A, Yamaki J, Homma Y. Characterization of circulating DNA in healthy human plasma. Clinica chimica acta; International Journal of Clinical Chemistry 2008; 387:55-8).

In one embodiment, any one of the methods for determining cf-DNA may be any one of the methods of U.S. Publication No. 2015-0086477-A1, and such methods are incorporated herein by reference in their entirety.

An amount of cf-DNA may also be determined by a MOMA assay. In one embodiment, any one of the methods for determining cf-DNA may be any one of the methods of PCT Publication No. WO 2016/176662 A1, and such methods are incorporated herein by reference in their entirety.

The cf-DNA, DS and/or total, may be determined using differences in sequence identity between the subject and donor genotype. Such differences may be single nucleotide variants (SNVs); however, wherever a SNV is referred to herein, any difference in sequence identity between recipient and donor-specific nucleic acids is intended to also be applicable. Thus, any one of the methods or compositions provided herein may be applied to recipient versus donor-specific nucleic acids where there is a difference in sequence identity. As used herein, “single nucleotide variant” refers to a nucleic acid sequence within which there is sequence variability, preferably in some embodiments at a single nucleotide. These SNVs include any mutations specific to or that can identify DS cf-DNA. Primers can be prepared as provided herein for any one or more of the SNVs.

The nucleic acid sequence within which there is sequence identity variability is generally referred to as a “target”. As used herein, a “SNV target” refers to a nucleic acid sequence within which there is sequence variability, such as at a single nucleotide. The SNV target has more than one allele, and in preferred embodiments, the SNV target is biallelic. Nucleic acids, such as donor-specific nucleic acids, can be quantified even at extremely low levels by performing amplification-based quantification assays, such as quantitative PCR assays, with primers specific for SNV targets. In some embodiments, the amount of nucleic acids is determined by attempting an amplification-based quantification assay, such as quantitative PCR, with primers for a plurality of SNV targets. A “plurality of SNV targets” refers to more than one SNV target where for each target there are at least two alleles. Preferably, in some embodiments, each SNV target is expected to be biallelic and a primer pair specific to each allele of the SNV target is used to specifically amplify nucleic acids of each allele, where amplification occurs if the nucleic acid of the specific allele is present in the sample. As used herein, one allele may be the donor-specific version of a target sequence and another allele is the recipient-specific version of the sequence.

In an embodiment of any one of the methods or compositions provided herein, one or more primer pairs for SNV target(s) can be pre-selected based on knowledge that the SNV targets will be informative, such as with knowledge of genotype, such as of the donor. In such embodiments, the genotype of the donor is known or can be determined. Thus, any one of the methods provided herein, can include a step of genotyping the donor or obtaining the donor genotype.

In other embodiments of any one of the methods provided herein, the genotype of the donor is unknown. In an embodiment of such cases, the donor genotype may be inferred with an expectation maximization method. As an example, using the known recipient genotype, targets known to be homozygous in the recipient can be selected. Any contaminants can be attributed to donor-specific nucleic acids, and the resulting assay collection will consist of a tri-modal distribution: non-, half-, and fully-informative assays. With a sufficient number of recipient homozygous assays, the presence of donor fully-informative assays can be inferred.

For example, if a recipient genotype is homozygous and known, then measurements not associated with the recipient genotype (those that are truly donor-homozygous) will have the highest cluster, and equal the guess (fully-informative), as compared to those that are donor-heterozygous, which will only be at half the guess (half-informative). Then, a probability distribution can be plotted and an expectation maximization algorithm (EM) can be used to infer donor genotype. The EM algorithm can be used to infer the donor genotype frequency in any one of the methods provided herein. Accordingly, an EM algorithm may be used to infer the most likely donor genotypes at all assayed SNV targets. Using inferred donor genotypes, quantification may proceed as in the full-information scenario discussed above. EM may begin on the assumption that the minor allele ratio found at an assay follows a tri-modal distribution (one for each combination of recipient (A) and donor (B), i.e., AA, AB, and BB). With all donor genotypes unknown, it is possible to bootstrap from the knowledge that any assays exhibiting nearly zero minor allele are donor AA (i.e., recipient alleles), and the highest is donor BB. Initial guesses for all donor genotypes may then be recorded, and the mean of each cluster can be calculated. Enforcing that the donor BB assays' mean is twice that of the donor AB restricts the search. The algorithm then reassigns guessed donor genotypes based on the clusters and built-in assumptions. The process is iterative until no more changes are made. The final result is a set of the most likely donor genotypes given their measured divergence from the background. Generally, every target falls into the model; a result may be excluded if between groups after maximization.

In another embodiment of any one of the methods or compositions provided herein, primer pairs for a plurality of SNV targets can be selected for the likelihood at least one (or more) may be informative. In such embodiments, primer pairs for a panel of SNV targets are used in any one of the methods provided herein. In some embodiments, the panel of SNV targets is a panel of at least 30, 35, 40, 45, 50, 55, 60, 65, 70, 75, 80, 85, 90, 95 or more possible targets.

As used herein, “an informative SNV target” is one in which amplification with primers as provided herein occurs, and the results of which are informative. “Informative results” as provided herein are the results that can be used to quantify the level of nucleic acids in a sample. In some embodiments, informative results exclude results that are considered “no call” or erroneous call results. From the informative results, allele percentages can be calculated using standard curves, in some embodiments of any one of the methods provided. In some embodiments of any one of the methods provided, the amount of donor-specific nucleic acids represents an average across informative results for the donor-specific nucleic acids, respectively.

The amount of nucleic acids may be determined with the quantities of the major and minor alleles as well as the genotype of the recipient in some embodiments. In some embodiments of any one of the methods provided herein, the alleles can be determined based on prior genotyping of the subject. Methods for genotyping are well known in the art. Such methods include sequencing, such as next generation, hybridization, microarray, other separation technologies or PCR assays. Any one of the methods provided herein can include steps of obtaining such genotypes.

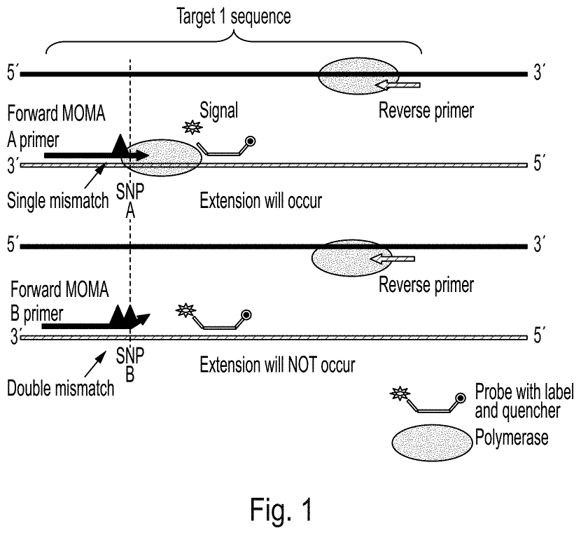

Primers for use in MOMA assays may be obtained, and any one of the methods provided herein can include a step of obtaining one or more primer pairs for performing the amplification-based quantification assays, such as PCR assays. Generally, the primers possess unique properties that facilitate their use in quantifying amounts of nucleic acids. For example, a forward primer of a primer pair can be mismatched at a 3′ nucleotide (e.g., penultimate 3′ nucleotide). In some embodiments of any one of the methods or compositions provided, this mismatch is at a 3′ nucleotide but adjacent to the SNV position. In some embodiments of any one of the methods or composition provided, the mismatch positioning of the primer relative to a SNV position is as shown in FIG. 1. Generally, such a forward primer, even with the 3′ mismatch, will produce an amplification product (in conjunction with a suitable reverse primer) in an amplification reaction, such as a PCR reaction, thus allowing for the amplification and resulting detection of a nucleic acid with the respective SNV. If the particular SNV is not present, and there is a double mismatch with respect to the other allele of the SNV target, an amplification product will generally not be produced. Preferably, in some embodiments of any one of the methods or compositions provided herein, for each SNV target, a primer pair is obtained whereby specific amplification of each allele can occur without amplification of the other allele(s). “Specific amplification” refers to the amplification of a specific allele of a target without substantial amplification of another nucleic acid or without amplification of another nucleic acid sequence above background or noise. In some embodiments, specific amplification results only in the amplification of the specific allele.

In some embodiments of any one of the methods or compositions provided herein, for each SNV target that is biallelic, there are two primer pairs, each specific to one of the two alleles and thus have a single mismatch with respect to the allele it is to amplify and a double mismatch with respect to the allele it is not to amplify (if nucleic acids of these alleles are present). In some embodiments of any one of the methods or compositions provided herein, the mismatch primer is the forward primer. In some embodiments of any one of the methods or compositions provided herein, the reverse primer of the two primer pairs for each SNV target is the same.

These concepts can be used in the design of primer pairs for any one of the methods and compositions provided herein. It should be appreciated that the forward and reverse primers are designed to bind opposite strands (e.g., a sense strand and an antisense strand) in order to amplify a fragment of a specific locus of the template. The forward and reverse primers of a primer pair may be designed to amplify a nucleic acid fragment of any suitable size to detect the presence of, for example, an allele of a SNV target according to the disclosure. Any one of the methods provided herein can include one or more steps for obtaining one or more primer pairs as described herein.

It should be appreciated that the primer pairs described herein may be used in a multiplex amplification-based quantification assay, such as a PCR assay. Accordingly, in some embodiments of any one of the methods or compositions provided herein, the primer pairs are designed to be compatible with other primer pairs in a PCR reaction. For example, the primer pairs may be designed to be compatible with at least 1, at least 2, at least 3, at least 4, at least 5, etc. other primer pairs in a PCR reaction. As used herein, primer pairs in a PCR reaction are “compatible” if they are capable of amplifying their target in the same PCR reaction. In some embodiments, primer pairs are compatible if the primer pairs are inhibited from amplifying their target DNA by no more than 1%, no more than 2%, no more than 3%, no more than 4%, no more than 5%, no more than 10%, no more than 15%, no more than 20%, no more than 25%, no more than 30%, no more than 35%, no more than 40%, no more than 45%, no more than 50%, or no more than 60% when multiplexed in the same PCR reaction. Primer pairs may not be compatible for a number of reasons including, but not limited to, the formation of primer dimers and binding to off-target sites on a template that may interfere with another primer pair. Accordingly, the primer pairs of the disclosure may be designed to prevent the formation of dimers with other primer pairs or limit the number of off-target binding sites. Exemplary methods for designing primers for use in a multiplex PCR assay are known in the art or otherwise described herein.

In some embodiments, the primer pairs described herein are used in a multiplex amplification-based quantification assay, such as a PCR assay, to quantify an amount of donor-specific nucleic acids. Accordingly, in some embodiments of any one of the methods or compositions provided herein, the primer pairs are designed to detect genomic regions that are diploid, excluding primer pairs that are designed to detect genomic regions that are potentially non-diploid. In some embodiments of any one of the methods or compositions provided herein, the primer pairs used in accordance with the disclosure do not detect repeat-masked regions, known copy-number variable regions, or other genomic regions that may be non-diploid.

In some embodiments of any one of the methods provided herein, the amplification-based quantitative assay is any quantitative assay, such as whereby nucleic acids are amplified and the amounts of the nucleic acids can be determined. Such assays include those whereby nucleic acids are amplified with the MOMA primers as described herein and quantified. Such assays include simple amplification and detection, hybridization techniques, separation technologies, such as electrophoresis, next generation sequencing and the like.

In some embodiments of any one of the methods provided herein the PCR is quantitative PCR meaning that amounts of nucleic acids can be determined. Quantitative PCR include real-time PCR, digital PCR, TAQMAN™, etc. In some embodiments of any one of the methods provided herein the PCR is “real-time PCR”. Such PCR refers to a PCR reaction where the reaction kinetics can be monitored in the liquid phase while the amplification process is still proceeding. In contrast to conventional PCR, real-time PCR offers the ability to simultaneously detect or quantify in an amplification reaction in real time. Based on the increase of the fluorescence intensity from a specific dye, the concentration of the target can be determined even before the amplification reaches its plateau.

The use of multiple probes can expand the capability of single-probe real-time PCR. Multiplex real-time PCR uses multiple probe-based assays, in which each assay can have a specific probe labeled with a unique fluorescent dye, resulting in different observed colors for each assay. Real-time PCR instruments can discriminate between the fluorescence generated from different dyes. Different probes can be labeled with different dyes that each have unique emission spectra. Spectral signals are collected with discrete optics, passed through a series of filter sets, and collected by an array of detectors. Spectral overlap between dyes may be corrected by using pure dye spectra to deconvolute the experimental data by matrix algebra.

A probe may be useful for methods of the present disclosure, particularly for those methods that include a quantification step. Any one of the methods provided herein can include the use of a probe in the performance of the PCR assay(s), while any one of the compositions or kits provided herein can include one or more probes. Importantly, in some embodiments of any one or more of the methods provided herein, the probe in one or more or all of the PCR quantification assays is on the same strand as the mismatch primer and not on the opposite strand. It has been found that in so incorporating the probe in a PCR reaction, additional allele specific discrimination can be provided. As an example, a TAQMAN™ probe is a hydrolysis probe that has a FAM™ or VIC® dye label on the 5′ end, and minor groove binder (MGB) non-fluorescent quencher (NFQ) on the 3′ end. The TAQMAN™ probe principle generally relies on the 5′-3′ exonuclease activity of Tag® polymerase to cleave the dual-labeled TAQMAN™ probe during hybridization to a complementary probe-binding region and fluorophore-based detection. TAQMAN™ probes can increase the specificity of detection in quantitative measurements during the exponential stages of a quantitative PCR reaction.

PCR systems generally rely upon the detection and quantitation of fluorescent dyes or reporters, the signal of which increase in direct proportion to the amount of PCR product in a reaction. For example, in the simplest and most economical format, that reporter can be the double-stranded DNA-specific dye SYBR® Green (Molecular Probes). SYBR® Green is a dye that binds the minor groove of double-stranded DNA. When SYBR® Green dye binds to a double-stranded DNA, the fluorescence intensity increases. As more double-stranded amplicons are produced, SYBR® Green dye signal will increase.

It should be appreciated that the PCR conditions provided herein may be modified or optimized to work in accordance with any one of the methods described herein. Typically, the PCR conditions are based on the enzyme used, the target template, and/or the primers. In some embodiments, one or more components of the PCR reaction is modified or optimized. Non-limiting examples of the components of a PCR reaction that may be optimized include the template DNA, the primers (e.g., forward primers and reverse primers), the deoxynucleotides (dNTPs), the polymerase, the magnesium concentration, the buffer, the probe (e.g., when performing real-time PCR), the buffer, and the reaction volume.

In any of the foregoing embodiments, any DNA polymerase (enzyme that catalyzes polymerization of DNA nucleotides into a DNA strand) may be utilized, including thermostable polymerases. Suitable polymerase enzymes will be known to those skilled in the art, and include E. coli DNA polymerase, Klenow fragment of E. coli DNA polymerase I, T7 DNA polymerase, T4 DNA polymerase, T5 DNA polymerase, Klenow class polymerases, Taq polymerase, Pfu DNA polymerase, Vent polymerase, bacteriophage 29, REDTaq™ Genomic DNA polymerase, or sequenase. Exemplary polymerases include, but are not limited to Bacillus stearothermophilus pol I, Thermus aquaticus (Taq) pol I, Pyrococcus furiosus (Pfu), Pyrococcus woesei (Pwo), Thermus flavus (Tfl), Thermus thermophilus (Tth), Thermus litoris (Tli) and Thermotoga maritime (Tma). These enzymes, modified versions of these enzymes, and combination of enzymes, are commercially available from vendors including Roche, Invitrogen, Qiagen, Stratagene, and Applied Biosystems. Representative enzymes include PHUSION® (New England Biolabs, Ipswich, Mass.), Hot MasterTaq™ (Eppendorf), PHUSION® Mpx (Finnzymes), PyroStart® (Fermentas), KOD (EMD Biosciences), Z-Taq (TAKARA), and CS3AC/LA (KlenTaq, University City, Mo.).

Salts and buffers include those familiar to those skilled in the art, including those comprising MgCl₁2, and Tris-HCl and KCl, respectively. Typically, 1.5-2.0 nM of magnesium is optimal for Taq DNA polymerase, however, the optimal magnesium concentration may depend on template, buffer, DNA and dNTPs as each has the potential to chelate magnesium. If the concentration of magnesium [Mg²⁺] is too low, a PCR product may not form. If the concentration of magnesium [Mg²⁺] is too high, undesired PCR products may be seen. In some embodiments the magnesium concentration may be optimized by supplementing magnesium concentration in 0.1 mM or 0.5 mM increments up to about 5 mM.

Buffers used in accordance with the disclosure may contain additives such as surfactants, dimethyl sulfoxide (DMSO), glycerol, bovine serum albumin (BSA) and polyethylene glycol (PEG), as well as others familiar to those skilled in the art. Nucleotides are generally deoxyribonucleoside triphosphates, such as deoxyadenosine triphosphate (dATP), deoxycytidine triphosphate (dCTP), deoxyguanosine triphosphate (dGTP), and deoxythymidine triphosphate (dTTP), which are also added to a reaction adequate amount for amplification of the target nucleic acid. In some embodiments, the concentration of one or more dNTPs (e.g., dATP, dCTP, dGTP, dTTP) is from about 10 μM to about 500 μM which may depend on the length and number of PCR products produced in a PCR reaction.

In some embodiments, the concentration of primers used in the PCR reaction may be modified or optimized. In some embodiments, the concentration of a primer (e.g., a forward or reverse primer) in a PCR reaction may be, for example, about 0.05 μM to about 1 μM. In particular embodiments, the concentration of each primer is about 1 nM to about 1 μM. It should be appreciated that the primers in accordance with the disclosure may be used at the same or different concentrations in a PCR reaction. For example, the forward primer of a primer pair may be used at a concentration of 0.5 μM and the reverse primer of the primer pair may be used at 0.1 μM. The concentration of the primer may be based on factors including, but not limited to, primer length, GC content, purity, mismatches with the target DNA or likelihood of forming primer dimers.

In some embodiments, the thermal profile of the PCR reaction is modified or optimized. Non-limiting examples of PCR thermal profile modifications include denaturation temperature and duration, annealing temperature and duration and extension time.

The temperature of the PCR reaction solutions may be sequentially cycled between a denaturing state, an annealing state, and an extension state for a predetermined number of cycles. The actual times and temperatures can be enzyme, primer, and target dependent. For any given reaction, denaturing states can range in certain embodiments from about 70° C. to about 100° C. In addition, the annealing temperature and time can influence the specificity and efficiency of primer binding to a particular locus within a target nucleic acid and may be important for particular PCR reactions. For any given reaction, annealing states can range in certain embodiments from about 20° C. to about 75° C. In some embodiments, the annealing state can be from about 46° C. to 64° C. In certain embodiments, the annealing state can be performed at room temperature (e.g., from about 20° C. to about 25° C.).

Extension temperature and time may also impact the allele product yield. For a given enzyme, extension states can range in certain embodiments from about 60° C. to about 75° C.

Quantification of the amounts of the alleles from a PCR assay can be performed as provided herein or as otherwise would be apparent to one of ordinary skill in the art. As an example, amplification traces are analyzed for consistency and robust quantification. Internal standards may be used to translate the cycle threshold to amount of input nucleic acids (e.g., DNA). The amounts of alleles can be computed as the mean of performant assays and can be adjusted for genotype.

Other methods for determining total cell-free DNA in a sample are known in the art. In some embodiments of any one of the methods provided herein, the total cell-free DNA is determined with TAQMAN™ Real-time PCR using RNase P as a target.

Any one of the methods provided herein can comprise extracting nucleic acids, such as cell-free DNA, from a sample obtained from a subject. Such extraction can be done using any method known in the art or as otherwise provided herein (see, e.g., Current Protocols in Molecular Biology, latest edition, or the QIAamp circulating nucleic acid kit or other appropriate commercially available kits). An exemplary method for isolating cell-free DNA from blood is described. Blood containing an anti-coagulant such as EDTA or DTA is collected from a subject. The plasma, which contains cf-DNA, is separated from cells present in the blood (e.g., by centrifugation or filtering). An optional secondary separation may be performed to remove any remaining cells from the plasma (e.g., a second centrifugation or filtering step). The cf-DNA can then be extracted using any method known in the art, e.g., using a commercial kit such as those produced by Qiagen. Other exemplary methods for extracting cf-DNA are also known in the art (see, e.g., Cell-Free Plasma DNA as a Predictor of Outcome in Severe Sepsis and Septic Shock. Clin. Chem. 2008, v. 54, p. 1000-1007; Prediction of MYCN Amplification in Neuroblastoma Using Serum DNA and Real-Time Quantitative Polymerase Chain Reaction. JCO 2005, v. 23, p. 5205-5210; Circulating Nucleic Acids in Blood of Healthy Male and Female Donors. Clin. Chem. 2005, v. 51, p. 1317-1319; Use of Magnetic Beads for Plasma Cell-free DNA Extraction: Toward Automation of Plasma DNA Analysis for Molecular Diagnostics. Clin. Chem. 2003, v. 49, p. 1953-1955; Chiu R W K, Poon L L M, Lau T K, Leung T N, Wong E M C, Lo Y M D. Effects of blood-processing protocols on fetal and total DNA quantification in maternal plasma. Clin Chem 2001; 47:1607-1613; and Swinkels et al. Effects of Blood-Processing Protocols on Cell-free DNA Quantification in Plasma. Clinical Chemistry, 2003, vol. 49, no. 3, 525-526).

In some embodiments of any one of the methods provided herein, a pre-amplification step is performed. An exemplary method of such an amplification is as follows, and such a method can be included in any one of the methods provided herein. Approximately 15 ng of cell-free plasma DNA is amplified in a PCR using Q5 DNA polymerase with approximately 13 targets where pooled primers were at 4uM total. Samples undergo approximately 25 cycles. Reactions are in 25 ul total. After amplification, samples can be cleaned up using several approaches including AMPURE bead cleanup, bead purification, or simply ExoSAP-IT™, or Zymo.

As used herein, the sample from a subject can be a biological sample. Examples of such biological samples include whole blood, plasma, serum, urine, etc. In some embodiments, addition of further nucleic acids, e.g., a standard, to the sample can be performed.

In another aspect, compositions and kits comprising one or more primer pairs as provided herein are provided. Other reagents for performing an assay, such as a PCR assay, may also be included in the composition or kit.

Various aspects of the present invention may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments of the invention may be implemented as one or more methods, of which an example has been provided. The acts performed as part of the method(s) may be ordered in any suitable way. Accordingly, embodiments may be constructed in which acts are performed in an order different from illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The following description provides examples of the methods provided herein.

### EXAMPLES

### Example 1

### MOMA Assay With Recipient and Donor Genotype Information

### SNV Target Selection

Identification of targets for multiplexing in accordance with the disclosure may include one or more of the following steps. First, highly heterozygous SNPs were screened on several ethnic control populations (Hardy-Weinberg p>0.25), excluding known difficult regions. Difficult regions include syndromic regions likely to be abnormal in patients and regions of low complexity, including centromeres and telomeres of chromosomes. Target fragments of desired lengths were then designed in silico. Specifically, two 20-26 bp primers spanning each SNP's 70 bp window were designed. All candidate primers were then queried to GCRh37 using BLAST. Those primers that were found to be sufficiently specific were retained, and monitored for off-target hits, particularly at the 3′ end of the fragment. The off-target candidate hits were analyzed for pairwise fragment generation that would survive size selection. Selected primers were then subjected to an in silico multiplexing evaluation. The primers' computed melting temperatures and guanine-cytosine percentages (GC %) were used to filter for moderate range sequences. An iterated genetic algorithm and simulated annealing were used to select candidate primers compatible for 400 targets, ultimately resulting in the selection of 800 primers. The 800 primers were generated and physically tested for multiplex capabilities at a common melting temperature in a common solution. Specifically, primers were filtered based on even amplification in the multiplex screen and moderate read depth window. Forty-eight assays were designed for MOMA using the top performing multiplexed SNPs. Each SNP had a probe designed in WT/MUT at four mismatch choices; there were eight probes per assay. The new nested primers were designed within the 70 bp enriched fragments. Finally, the primers were experimentally amplified with known heterozygous individuals to evaluate amplification efficiency (8 probes×48 assays in triplicate, using TAQMAN™).

A priori Genotyping Informativeness of each Assay

Using the known recipient and donor genotypes at each assayed SNP, a subset of informative assays was selected. Note that recipient homozygous sites can be used where the donor is any other genotype. Additionally, if the donor genotype is not known, it can be inferred, such as by using plasma data discrepancies. Genotypes may also be learned through sequencing, SNP microarray, or application of a MOMA assay on known 0% (clean recipient) samples.

### Post Processing Analysis of Multiplex Assay Performance

Patient-specific MOMA probe biases were estimated across the experimental cohort. Selection iteratively was refined to make the final donor percent call. Further, automatic outlier detection provided patient-specific anomalous genomic regions.

### Reconstruction Experiment

The sensitivity and precision of the assay were evaluated using reconstructed plasma samples with known mixing ratios. Specifically, the ratios of 1:10, 1:20, 1:100, 1:200, and 1:1000 were evaluated.

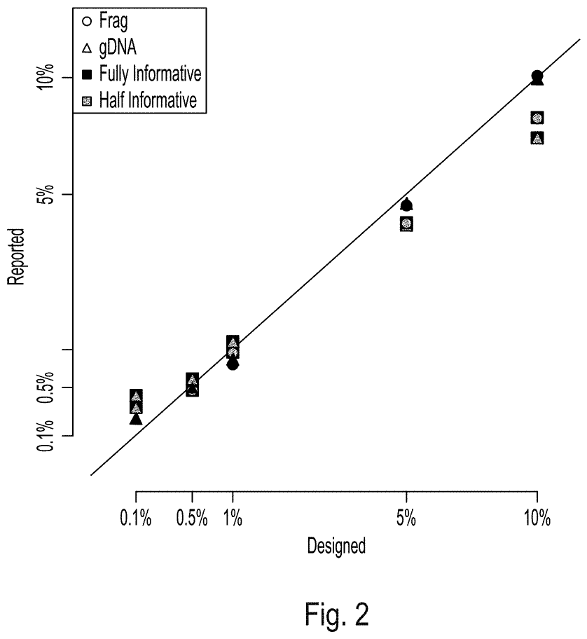

Results of the reconstruction experiment are shown in FIG. 2. One target is fully informative where there is a homozygous donor against a homozygous recipient (shaded data points). The other target is half informative where there is a heterozygous donor against a homozygous recipient (open data points).

### Example 2

### MOMA Assay with Recipient but not Donor Genotype Information

To work without donor genotype information, the following procedure may be performed to infer informative assays and allow for quantification of donor-specific cell-free DNA in plasma samples. All assays were evaluated for performance in the full information scenario. This procedure thus assumed clean AA/AB/BB genotypes at each assay and unbiased behavior of each quantification. With recipient genotype, assays known to be homozygous in the recipient were selected. Any contamination was attributed to the donor nucleic acids, and the assay collection created a tri-modal distribution with three clusters of assays corresponding to the non-, half, and fully-informative assays. With sufficient numbers of recipient homozygous assays, the presence of donor fully informative assays can be assumed.

If the recipient genotype is homozygous and known, then if a measurement that is not the recipient genotype is observed, the probes which are truly donor-homozygous will have the highest cluster and equal the guess whereas those that are donor heterozygous will be at half the guess. A probability distribution can be plotted and an expectation maximization algorithm (EM) can be employed to infer donor genotype. Such can be used to infer the donor genotype frequency in any one of the methods provided herein. Accordingly, an EM algorithm was used to infer the most likely donor genotypes at all assayed SNV targets. With inferred donor genotypes, quantification may proceed as in the full-information scenario. EM can begin with the assumption that the minor allele ratio found at an assay follows a tri-modal distribution, one for each combination of recipient and donor, given all assays are “AA” in the recipient (or flipped from “BB” without loss of generality). With all donor genotypes unknown, it is possible to bootstrap from the knowledge that any assays exhibiting nearly zero minor allele are donor AA, and the highest is donor BB. Initial guesses for all donor genotypes were recorded, and the mean of each cluster calculated. Enforcing that the donor BB assays' mean is twice that of the donor AB restricts the search. The algorithm then reassigns guessed donor genotypes based on the clusters and built-in assumptions. The process was iterative until no more changes were made. The final result is a set of the most likely donor genotypes given their measured divergence from the background. Generally, every target falls into the model; a result may be tossed if between groups after maximization.

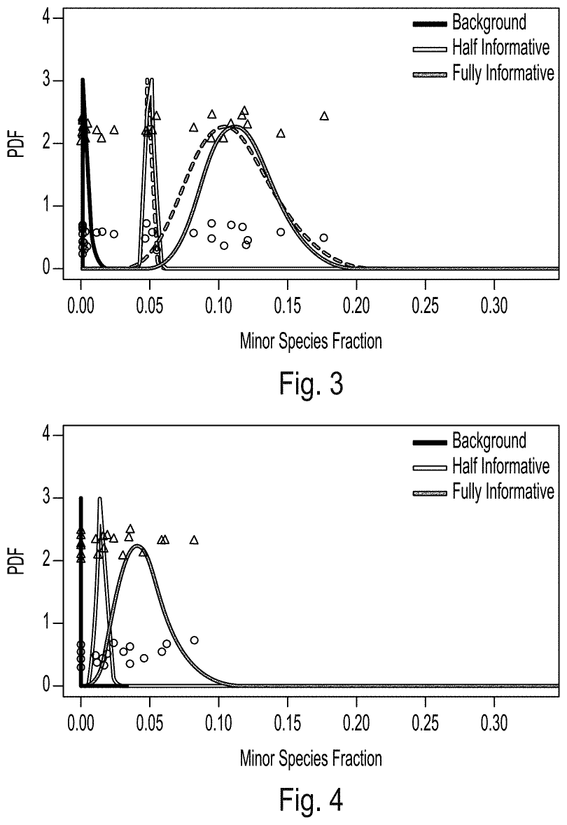

FIGS. 3 and 4 show exemplary results from plasma samples handled in this manner. The x-axis is the donor % for any assay found recipient homozygous. The rows of points represent individual PCR assay results. The bottom-most row of circles represents the initial guess of donor genotypes, some AA, some A/B and some BB. Then the solid curves were drawn representing beta distributions centered on the initial assays, spotted for homozygous (fully informative) and white for heterozygous (half informative) with black curves representing the distribution of non-informative assays or background noise. The assays were re-assigned updated guesses in the second row. The second row's curves use dashed lines. The top row is the final estimate because no change occurred. Double the peak of the white dashed curve corresponds to the maximum likelihood donor % call, at around 10%, or equal to the mean of the dotted curve.

A reconstruction experiment (Recon1) using DNA from two individuals was created at 10%, 5%, 1%, 0.5%, and 0.1%. All mixes were amplified with a multiplex library of targets, cleaned, then quantitatively genotyped using a MOMA method. The analysis was performed with genotyping each individual in order to know their true genotypes. Informative targets were determined using prior knowledge of the genotype of the major individual (looking for homozygous sites) and where the second individual was different, and used to calculate fractions (percentage) using informative targets. The fractions were then calculated (depicted in black to denote “With Genotype” information).

A second reconstruction experiment (Recon2), beginning with two individuals, major and minor, was also created at 10%, 5%, 1%, 0.5%, and 0.1%. All mixes were amplified with the multiplex library of targets, cleaned, and then quantitatively genotyped using a MOMA method. The analysis was performed by genotyping each individual in order to know their true genotypes. Informative targets were determined using prior knowledge of the genotype of the second individual as described above. The fractions were then calculated (depicted in black to denote “With Genotype” information).

These reconstructions were run again the next day (Recon3).

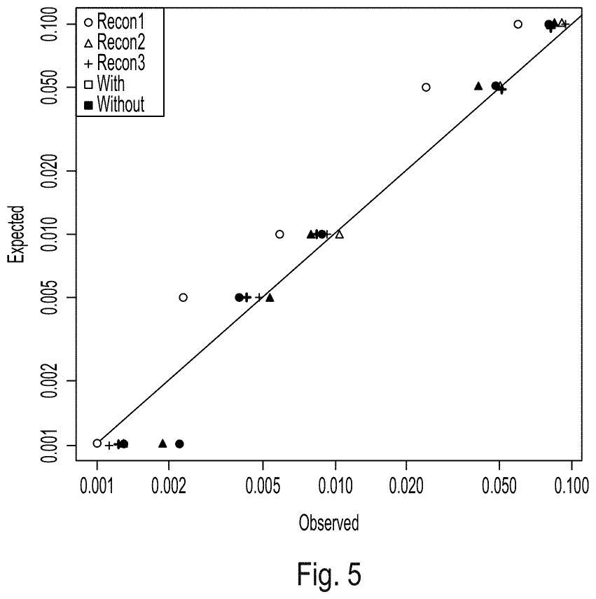

The same reconstruction samples (Recon 1, 2, 3) were then analyzed again only using the genotyping information available for the first individual (major DNA contributor). Genotyping information from the second individual (minor DNA contributor) was not used. Approximately 38-40 targets were used to calculate fractions without genotyping (simulating without donor); they are presented as shaded points (FIG. 5). It was found that each target that was recipient homozygous was generally useful. The circles show a first estimate, a thresholding; those on the right were thought to be fully informative and those on the left, not. The triangles along the top were the same targets, but for the final informativity decisions they were recolored.

### Example 3

### MOMA cf-DNA Assay

Principles and Procedures of a MOMA cf-DNA Assay

This exemplary assay is designed to determine the percentage of DS cf-DNA present in a transplant recipient's blood sample. In this embodiment, the recipient's blood sample is collected in an EDTA tube and centrifuged to separate the plasma and buffy coat. The plasma and buffy coat can be aliquoted into two separate 15 mL conical tubes and frozen. The plasma sample can be used for quantitative genotyping (qGT), while the buffy coat can be used for basic genotyping (bGT) of the recipient. In addition to the transplant recipient's blood sample, a small piece of discarded tissue or blood sample from the donor can be used for basic genotyping.

The first step in the process can be to extract cell free DNA from the plasma sample (used for qGT) and genomic DNA (gDNA) from the buffy coat, whole blood, or tissue sample (used for bGT). The total amount of cfDNA can be determined by qPCR and normalized to a target concentration. This process is known as a cfDNA Quantification. gDNA can be quantified using UV-spectrophotometry and normalized. Fifteen ng of DNA generally provides accurate and valid results.

The normalized patient DNA can be used as an input into a highly-multiplexed library PCR amplification reaction containing, for example, 96 primer pairs, each of which amplify a region including one of the MOMA target sites. The resulting library can be used as the input for either the bGT or qGT assay as it consists of PCR amplicons having the MOMA target primer and probe sites. This step can improve the sensitivity of the overall assay by increasing the copy number of each target prior to the highly-specific qPCR amplification. Controls and calibrators/standards can be amplified with the multiplex library alongside patient samples. Following the library amplification, an enzymatic cleanup can be performed to remove excess primers and unincorporated deoxynucleotide triphosphates (dNTPs) to prevent interference with the downstream amplification.

In a parallel workflow the master mixes can be prepared and transferred to a 384-well PCR plate. The amplified samples, controls, and calibrators/standards can then be diluted with the library dilution buffer to a predetermined volume and concentration. The diluted samples and controls can be aliquoted to a 6-well reservoir plate and transferred to the 384-well PCR plate using an acoustic liquid handler. The plate can then be sealed and moved to a real-time PCR amplification and detection system.

MOMA can perform both the basic and quantitative genotyping analyses by targeting biallelic SNPs that are likely to be distinct between a transplant donor and recipient making them highly informative. The basic genotyping analysis can label the recipient and donor with three possible genotypes at each target (e.g. homozygous REF, heterozygous REF and VAR, and homozygous VAR). This information can be used for the quantitative genotyping analysis, along with standard curves, to quantitate to the allele ratio for each target, known as a minor-species proportion. The median of all informative and quality-control passed allele ratios can be used to determine the % of DS cfDNA.

### Example 4

### Examples of Computer-Implemented Embodiments

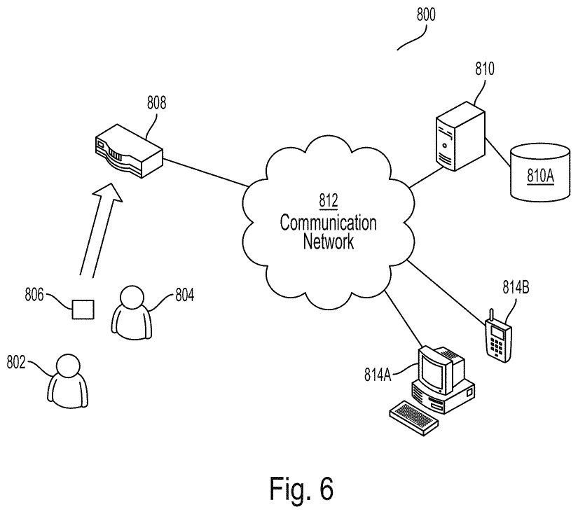

In some embodiments, the diagnostic techniques described above may be implemented via one or more computing devices executing one or more software facilities to analyze samples for a subject, such as over time, measure nucleic acids (such as cell-free DNA) in the samples, and produce a result, such as a diagnostic result, based on one or more of the samples. FIG. 6 illustrates an example of a computer system with which some embodiments may operate, though it should be appreciated that embodiments are not limited to operating with a system of the type illustrated in FIG. 6.

The computer system of FIG. 6 includes a subject 802 and a clinician 804 that may obtain a 806 from the subject 806. As should be appreciated from the foregoing, the sample 806 may be any suitable sample of biological material for the subject 802 that may be used to measure the presence of nucleic acids (such as cell-free DNA) in the subject 802, including a blood sample. The sample 806 may be provided to an analysis device 808, which one of ordinary skill will appreciate from the foregoing will analyze the sample 808 so as to determine (including estimate) amounts of nucleic acids (such as cell-free DNA), including amounts of DS nucleic acids (such as DS cell-free DNA) and/or total nucleic acids (such as total cf-DNA) and/or the product thereof in the sample 806 and/or the subject 802. For ease of illustration, the analysis device 808 is depicted as single device, but it should be appreciated that analysis device 808 may take any suitable form and may, in some embodiments, be implemented as multiple devices. To determine the amounts of nucleic acids (such as cell-free DNA) and/or the product thereof in the sample 806 and/or subject 802, the analysis device 808 may perform any of the techniques described above, and is not limited to performing any particular analysis. The analysis device 808 may include one or more processors to execute an analysis facility implemented in software, which may drive the processor(s) to operate other hardware and receive the results of tasks performed by the other hardware to determine on overall result of the analysis, which may be the amounts of nucleic acids (such as cell-free DNA) and/or the product thereof in the sample 806 and/or the subject 802. The analysis facility may be stored in one or more computer-readable storage media, such as a memory of the device 808. In other embodiments, techniques described herein for analyzing a sample may be partially or entirely implemented in one or more special-purpose computer components such as Application Specific Integrated Circuits (ASICs), or through any other suitable form of computer component that may take the place of a software implementation.

In some embodiments, the clinician 804 may directly provide the sample 806 to the analysis device 808 and may operate the device 808 in addition to obtaining the sample 806 from the subject 802, while in other embodiments the device 808 may be located geographically remote from the clinician 804 and subject 802 and the sample 806 may need to be shipped or otherwise transferred to a location of the analysis device 808. The sample 806 may in some embodiments be provided to the analysis device 808 together with (e.g., input via any suitable interface) an identifier for the sample 806 and/or the subject 802, for a date and/or time at which the sample 806 was obtained, or other information describing or identifying the sample 806.

The analysis device 808 may in some embodiments be configured to provide a result of the analysis performed on the sample 806 to a computing device 810, which may include a data store 810A that may be implemented as a database or other suitable data store. The computing device 810 may in some embodiments be implemented as one or more servers, including as one or more physical and/or virtual machines of a distributed computing platform such as a cloud service provider. In other embodiments, the device 810 may be implemented as a desktop or laptop personal computer, a smart mobile phone, a tablet computer, a special-purpose hardware device, or other computing device.

In some embodiments, the analysis device 808 may communicate the result of its analysis to the device 810 via one or more wired and/or wireless, local and/or wide-area computer communication networks, including the Internet. The result of the analysis may be communicated using any suitable protocol and may be communicated together with the information describing or identifying the sample 806, such as an identifier for the sample 806 and/or subject 802 or a date and/or time the sample 806 was obtained.

The computing device 810 may include one or more processors to execute a diagnostic facility implemented in software, which may drive the processor(s) to perform diagnostic techniques described herein. The diagnostic facility may be stored in one or more computer-readable storage media, such as a memory of the device 810. In other embodiments, techniques described herein for analyzing a sample may be partially or entirely implemented in one or more special-purpose computer components such as Application Specific Integrated Circuits (ASICs), or through any other suitable form of computer component that may take the place of a software implementation.

The diagnostic facility may receive the result of the analysis and the information describing or identifying the sample 806 and may store that information in the data store 810 A. The information may be stored in the data store 810A in association with other information for the subject 802, such as in a case that information regarding prior samples for the subject 802 was previously received and stored by the diagnostic facility. The information regarding multiple samples may be associated using a common identifier, such as an identifier for the subject 802. In some cases, the data store 810A may include information for multiple different subjects.

The diagnostic facility may also be operated to analyze results of the analysis of one or more samples 806 for a particular subject 802, identified by user input, so as to determine a diagnosis for the subject 802. The diagnosis may be a conclusion of a risk that the subject 802 has, may have, or may in the future develop a particular condition. The diagnostic facility may determine the diagnosis using any of the various examples described above, including by comparing the amounts of nucleic acids (such as cell-free DNA) and/or the product thereof determined for a particular sample 806 to one or more thresholds or by comparing a change over time in the amounts of nucleic acids (such as cell-free DNA) and/or the product thereof determined for samples 806 over time, such as to one or more thresholds. For example, the diagnostic facility may determine a risk to the subject 802 of a condition by comparing an amount of nucleic acids (such as cell-free DNA) and/or the product thereof for one or more samples 806 to one threshold and comparing an amount of nucleic acids (such as cell-free DNA) and/or the product thereof for the same sample(s) 806 to another threshold. Based on the comparisons to the thresholds, the diagnostic facility may produce an output indicative of a risk to the subject 802 of a condition.

As should be appreciated from the foregoing, in some embodiments, the diagnostic facility may be configured with different thresholds to which amounts of nucleic acids (such as cell-free DNA) and/or the product thereof may be compared. The different thresholds may, for example, correspond to different demographic groups (age, gender, race, economic class, presence or absence of a particular procedure/condition/other in medical history, or other demographic categories), different conditions, and/or other parameters or combinations of parameters. In such embodiments, the diagnostic facility may be configured to select thresholds against which amounts of nucleic acids (such as cell-free DNA) and/or the product thereof are to be compared, with different thresholds stored in memory of the computing device 810. The selection may thus be based on demographic information for the subject 802 in embodiments in which thresholds differ based on demographic group, and in these cases demographic information for the subject 802 may be provided to the diagnostic facility or retrieved (from another computing device, or a data store that may be the same or different from the data store 810A, or from any other suitable source) by the diagnostic facility using an identifier for the subject 802. The selection may additionally or alternatively be based on the condition for which a risk is to be determined, and the diagnostic facility may prior to determining the risk receive as input a condition and use the condition to select the thresholds on which to base the determination of risk. It should be appreciated that the diagnostic facility is not limited to selecting thresholds in any particular manner, in embodiments in which multiple thresholds are supported.

In some embodiments, the diagnostic facility may be configured to output for presentation to a user a user interface that includes a diagnosis of a risk and/or a basis for the diagnosis for a subject 802. The basis for the diagnosis may include, for example, amounts of nucleic acids (such as cell-free DNA) and/or the product thereof detected in one or more samples 806 for a subject 802. In some embodiments, user interfaces may include any of the examples of results, values, amounts, graphs, etc. discussed above. They can include results, values, amounts, etc. over time. For example, in some embodiments, a user interface may incorporate a graph similar to that shown in any one of the figures provided herein. In such a case, in some cases the graph may be annotated to indicate to a user how different regions of the graph may correspond to different diagnoses that may be produced from an analysis of data displayed in the graph. For example, thresholds against which the graphed data may be compared to determine the analysis may be imposed on the graph(s).

A user interface including a graph, particularly with the lines and/or shading, may provide a user with a far more intuitive and faster-to-review interface to determine a risk of the subject 802 based on amounts of nucleic acids (such as cell-free DNA) and/or the product thereof, than may be provided through other user interfaces. It should be appreciated, however, that embodiments are not limited to being implemented with any particular user interface.

In some embodiments, the diagnostic facility may output the diagnosis or a user interface to one or more other computing devices 814 (including devices 814A, 814B) that may be operated by the subject 802 and/or a clinician, which may be the clinician 804 or another clinician. The diagnostic facility may transmit the diagnosis and/or user interface to the device 814 via the network(s) 812.

Techniques operating according to the principles described herein may be implemented in any suitable manner. Included in the discussion above are a series of flow charts showing the steps and acts of various processes that determine a risk of a condition based on an analysis of amounts of nucleic acids (such as cell-free DNA). The processing and decision blocks discussed above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that embodiments are not limited to any particular syntax or operation of any particular circuit or of any particular programming language or type of programming language. Rather, one skilled in the art may use the description above to fabricate circuits or to implement computer software algorithms to perform the processing of a particular apparatus carrying out the types of techniques described herein. It should also be appreciated that, unless otherwise indicated herein, the particular sequence of steps and/or acts described above is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as a portion of a computing device or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computer system of FIG. 6, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing device (co-located or geographically distributed) dedicated to executing the techniques described herein, one or more Field-Programmable Gate Arrays (FPGAs) for carrying out the techniques described herein, or any other suitable system.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Any one of the aforementioned, including the aforementioned devices, systems, embodiments, methods, techniques, algorithms, media, hardware, software, interfaces, processors, displays, networks, inputs, outputs or any combination thereof are provided herein in other aspects.

### Example 6

### Receiver Operating Characteristic (ROC) on Repeated Measures Using Correlation

Clinical information was collected including patient demographics and clinical data throughout the admission for transplant, around treatment episodes for rejection, around all symptomatic and asymptomatic biopsies, and around all hospital readmissions. Date and exact time of first biopsy were checked against blood sample date and time to ensure that all analyzed blood samples were taken prior to any intra-cardiac access, as we have shown that the local trauma of biopsy leads to an acute elevation of DF-cfDNA.

Local reads for endomyocardial biopsy, echocardiograms, and coronary angiography performed for clinical purposes during the study period were recorded. Dates and times of critical events including treatment for infection, treatment for rejection, cardiac arrest, cardiac re-transplantation, initiation of mechanical circulatory support, death and last follow-up were recorded. Treatment for infection was defined as initiation of an anti-infective medication, or escalation of prophylactic medication to therapeutic dosing, for the purpose of treating suspected or proven infection. Treatment of rejection was defined as the first change in immunosuppressive therapy with the intent to treat suspected or biopsy-proven allograft rejection as documented in the medical record. Initiation of treatment for rejection was recorded as the date and time that the first dose of medication for treatment was administered to the patient. Mechanical circulatory support (MCS) was defined as either temporary or durable ventricular assist device, aortic balloon pump, total artificial heart, or extra-corporeal circulatory support. If a subject was diagnosed with cancer or post-transplant lymphoproliferative disease, or became pregnant, the first dates of diagnosis were recorded, and they were excluded from analysis as these conditions introduce a confounding source of additional “non-self” cell-free DNA. The pathology reports of all biopsies were reviewed and 2004 ISHLT grade was recorded, with biopsy-proven ACR defined as Grade 1R or higher and biopsy-proven AMR defined as any grading higher than pAMR 0. Results of all coronary angiography during the study period were recorded as graded according to the 2010 ISHLT grading system (Mehra et al 2010 JHLT) with a CAV grade ≥1 being defined as the presence of CAV.

Samples with total cell-free DNA (cf-DNA) and donor fraction cf-DNA were used for analysis. In total, there were 197 subjects having biopsy samples with both total cf-DNA and donor fraction cf-DNA data available. The 197 subjects had 1150 samples in total (biopsy and non-biopsy), which were used to analyze outcomes other than rejection. For rejection analysis, 824 samples (biopsy associated) from the 197 subjects were used for analysis.

Comorbidities were defined as follows: death, samples within 30 days prior to death; cardiac arrest, last sample before cardiac arrest; MCS, samples within one day prior through duration of MS; treatment for infection, samples within 14 days of treatment initiation; cancer, samples from patients with cancer, acute cellular rejection (ACR), grade of cellular rejection 1 (CR1) or higher; antibody-mediated rejection (AMR), grade of pAMR1 or higher; and graft vasculopathy, samples from patients with graft vasculopathy. Heathy (control) subjects had none of the above comorbidities.

No death-related samples were available in the plasma group, so the results were obtained by combining the whole blood and plasma samples together, and calculating the receiver operating characteristic (ROC) curves overall, using the last sample per subject, and split into pediatric and adult subgroups.

Samples that were not healthy (e.g., had any of the comorbidities described above) were excluded from the control group. Samples within seven days post-transplant were also excluded. After these exclusions, the total number of samples available was 1073. A generalized linear model for repeated measures was used (subjects were clustered using covariance structures, as appropriate) with the log link function.

Death vs. Healthy (Control) Group—Total Cell free DNA (cf-DNA)

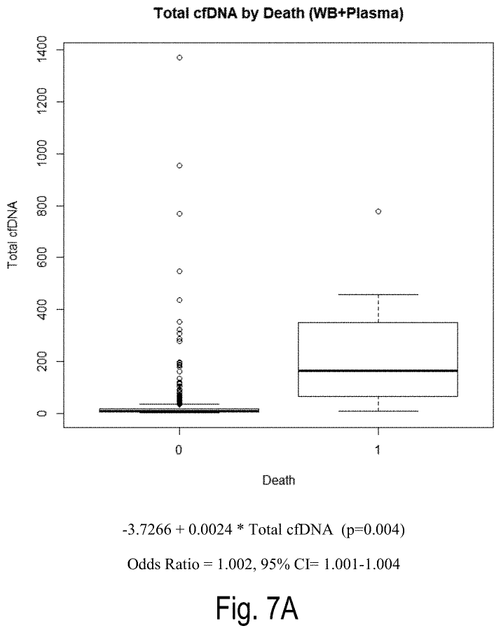

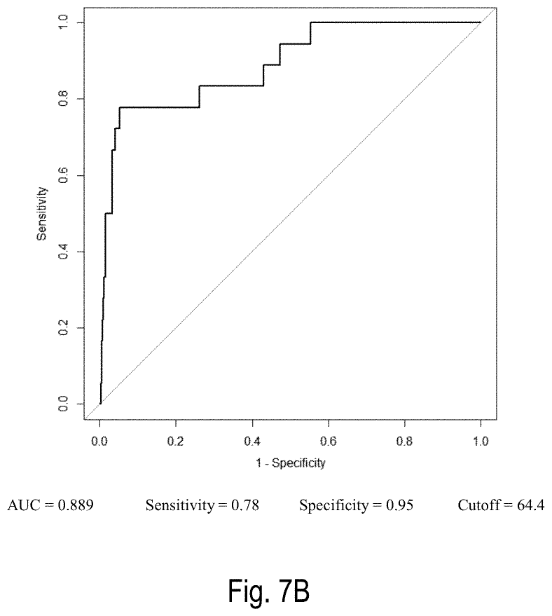

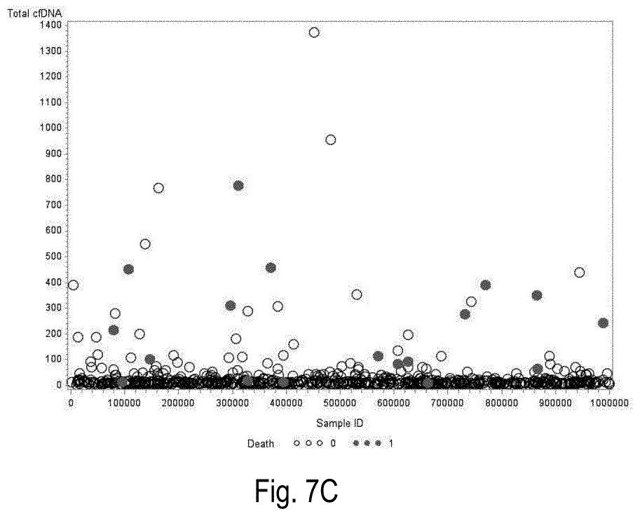

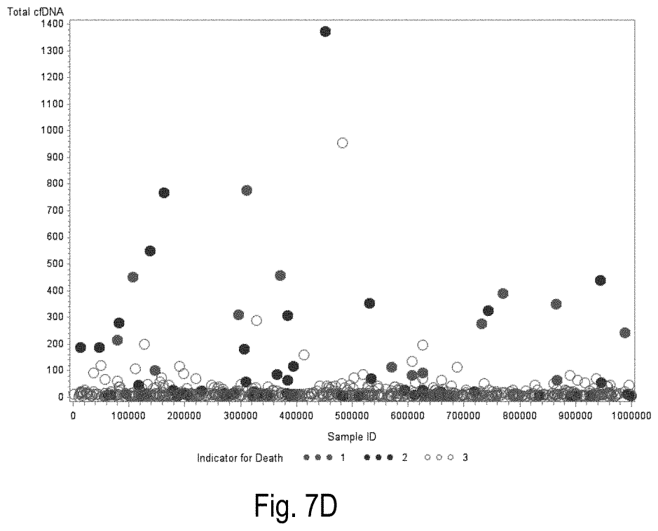

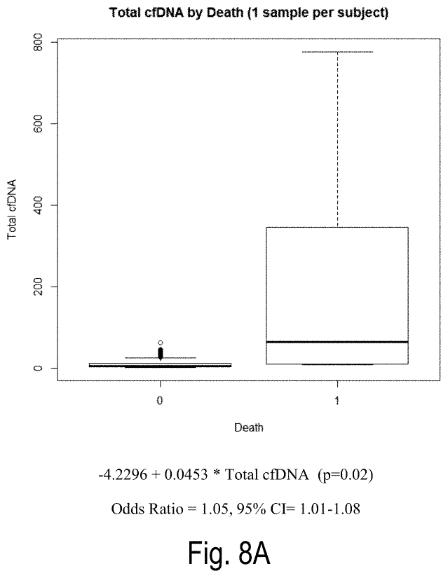

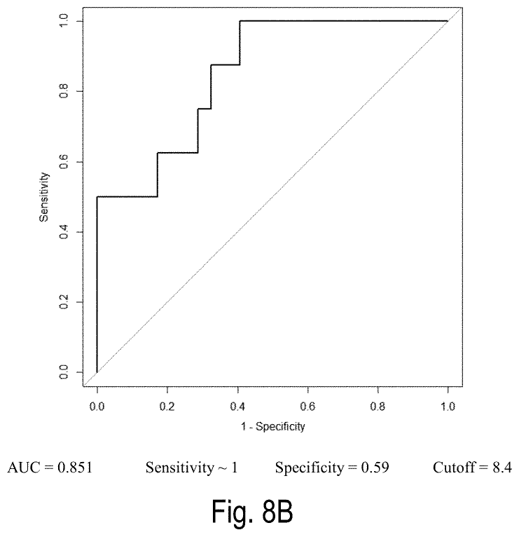

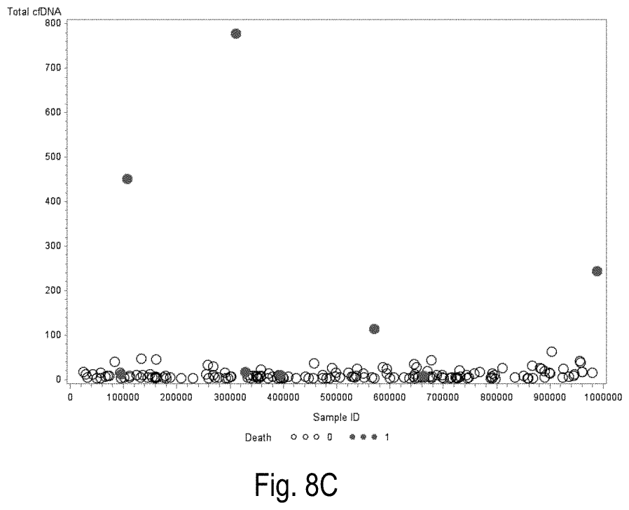

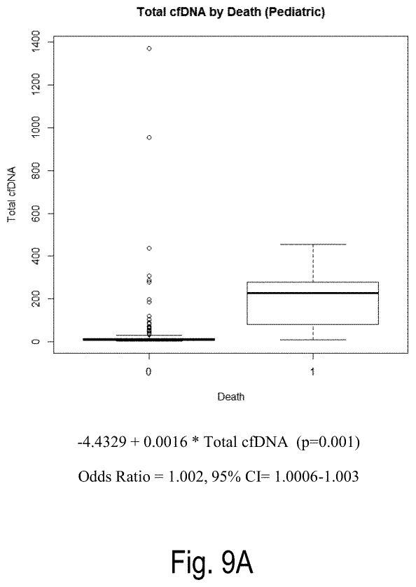

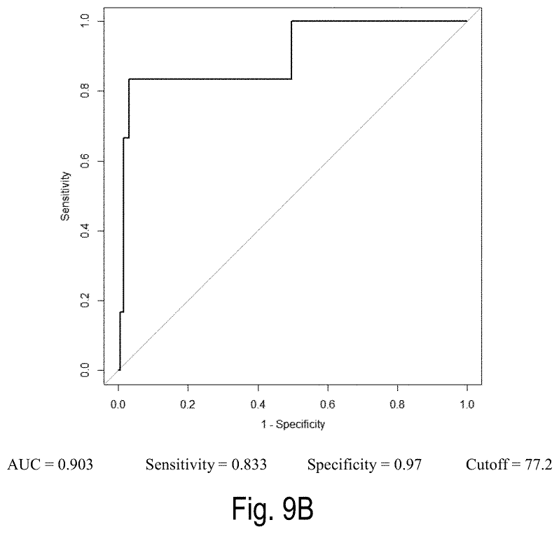

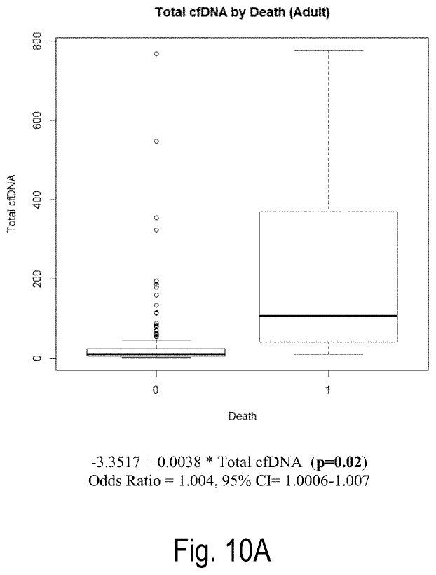

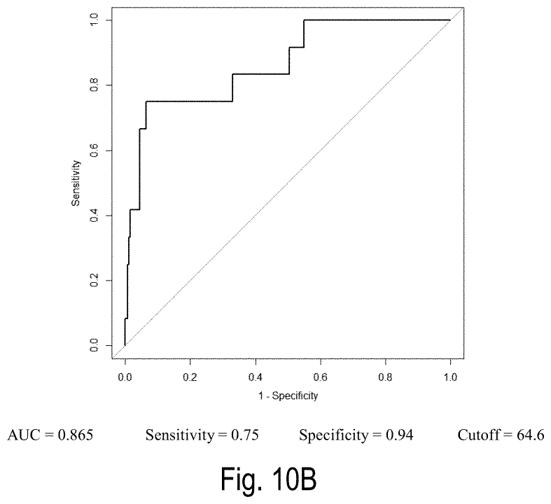

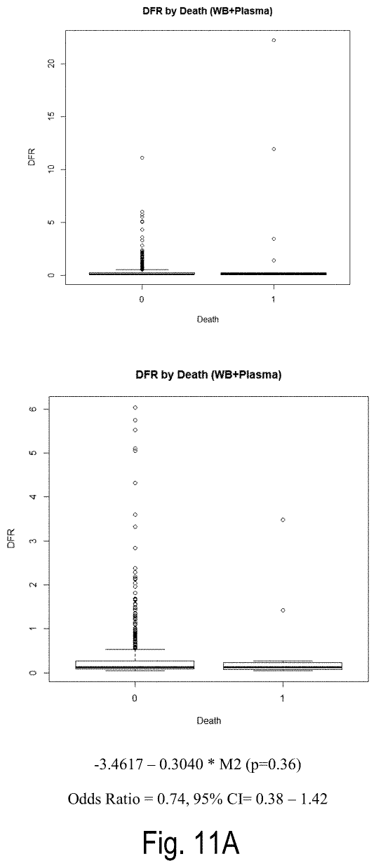

The data was analyzed to examine the total cell-free DNA (cf-DNA) levels in different samples of subjects who were healthy (e.g., had none of the comorbidities listed above) and those that died. Of the subjects who died, healthy samples not related to death (i.e., samples drawn more than 30 days before death) were included in the healthy group. The “not healthy” group excluded samples from those who did not die as well as samples obtained within 7 days post-transplant. The data is shown in FIGS. 7A-7D (all subjects), 8A-8C (the last sample from each subject), FIGS. 9A-9B (pediatric subjects), FIGS. 10A-10B (adult subjects), and in Tables 1-4 below.

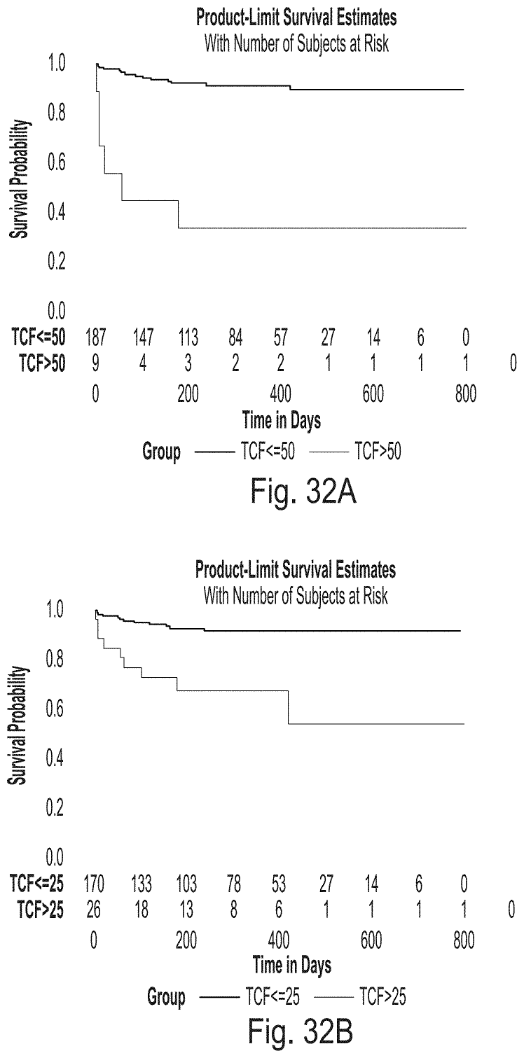

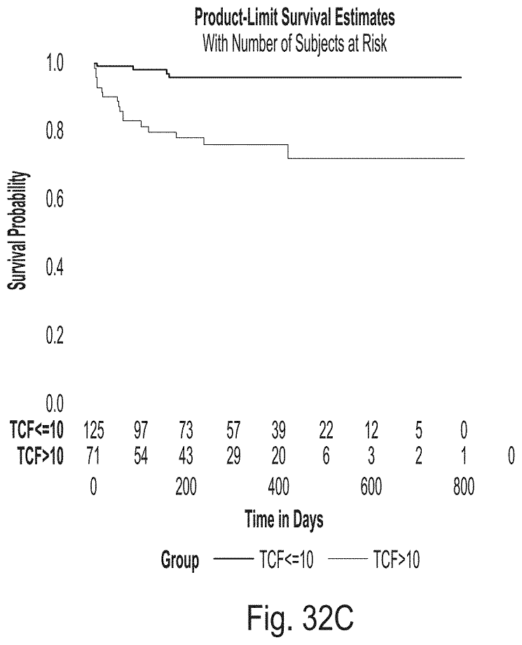

The data was further analyzed, using three different cutpoints: 50 ng/ml (FIG. 32A), 25 ng/ml (FIG. 32B), and 10 ng.ml (FIG. 32C). The statistics of the analysis are summarized in Table 5 below.

TABLE 1
Analysis of Plasma and Whole Blood Samples (Death vs. Healthy Group) - All Samples
Analysis Variable: plasma (ng/ml Plasma)

| | | | | | Lower | Upper | | |
|---|---|---|---|---|---|---|---|---|
| death | N | Mean | Std Dev | Median | Quartile | Quartile | Minimum | Maximum |
| 0 | 652 | 25.10 | 83.13 | 9.89 | 5.09 | 17.52 | 1.92 | 1372.10 |
| 1 | 18 | 220.51 | 207.99 | 163.97 | 65.03 | 349.26 | 8.55 | 777.08 |

TABLE 2
Analysis of Plasma and Whole Blood Samples (Death vs.
Healthy Group) - Last Sample per Subject
Analysis Variable: plasma (ng/ml Plasma)

| | | | | | Lower | Upper | | |
|---|---|---|---|---|---|---|---|---|
| death | N | Mean | Std Dev | Median | Quartile | Quartile | Minimum | Maximum |
| 0 | 157 | 10.70 | 10.70 | 6.06 | 3.93 | 13.36 | 1.92 | 62.92 |
| 1 | 8 | 203.90 | 279.43 | 65.24 | 10.94 | 346.61 | 8.55 | 777.08 |

TABLE 3
Analysis of Plasma and Whole Blood Samples (Death vs.
Healthy Group) - Pediatric Subjects
Analysis Variable: plasma ng/ml Plasma Total cfDNA

| | | | | | Lower | Upper | | |
|---|---|---|---|---|---|---|---|---|
| death | N | Mean | Std Dev | Median | Quartile | Quartile | Minimum | Maximum |
| 0 | 388 | 23.15 | 91.73 | 8.51 | 4.71 | 15.24 | 2.17 | 1372.10 |
| 1 | 6 | 213.39 | 156.99 | 228.57 | 81.53 | 276.91 | 8.55 | 456.24 |

TABLE 4
Analysis of Plasma and Whole Blood Samples (Death vs.
Healthy Group) - Adult Subjects
Analysis Variable: plasma Total cfDNA

| | | | | | Lower | Upper | | |
|---|---|---|---|---|---|---|---|---|
| death | N | Mean | Std Dev | Median | Quartile | Quartile | Minimum | Maximum |
| 0 | 264 | 27.96 | 68.62 | 11.50 | 5.76 | 24.32 | 1.92 | 768.47 |
| 1 | 12 | 224.06 | 235.82 | 107.24 | 40.89 | 369.91 | 10.46 | 777.08 |

TABLE 5
Analysis of Total Cell-free DNA and Relationship to Death

| Total cfDNA | >50 ng/ml | >25 ng/ml | >10 ng/ml |
|---|---|---|---|
| Hazard Ratio | 12.10 | 5.44 | 7.63 |
| 95% Conf Interval | 4.67-31.30 | 2.29-12.92 | 2.57-22.67 |
| NPV | 92% | 93% | 97% |
| Specificity | 98% | 90% | 69% |

Death vs. Healthy (Control) Group—Donor Fraction Cell free DNA (cf-DNA)

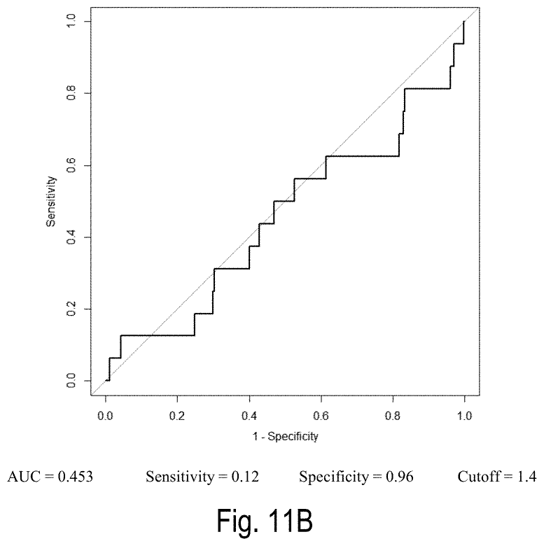

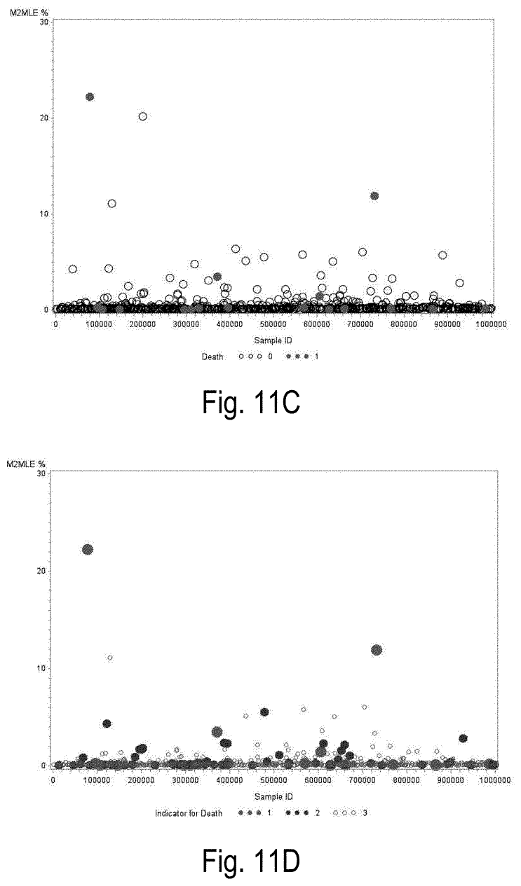

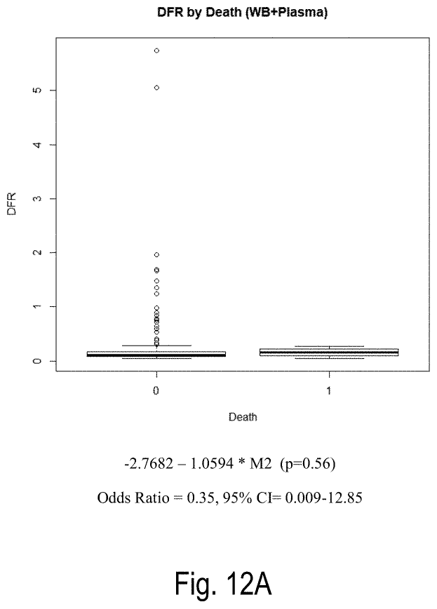

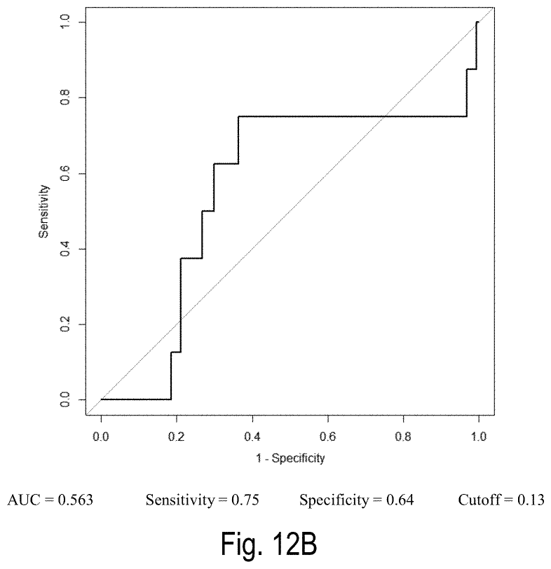

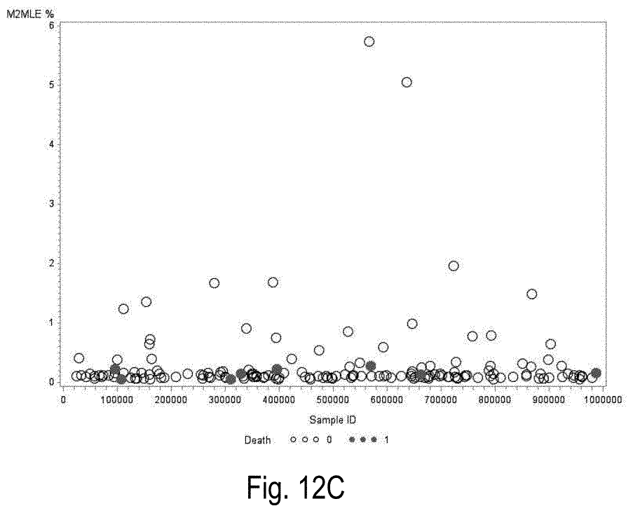

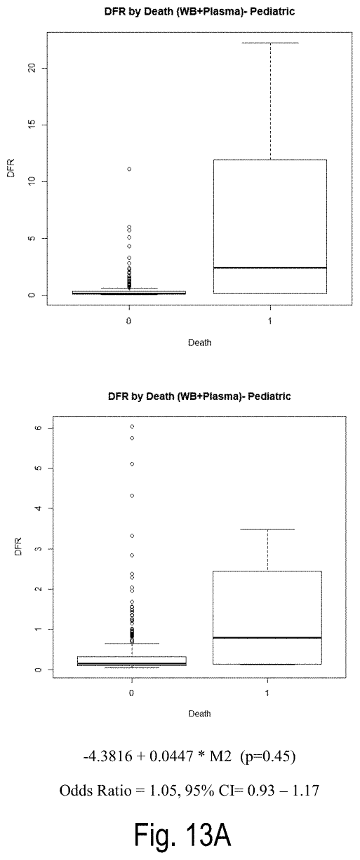

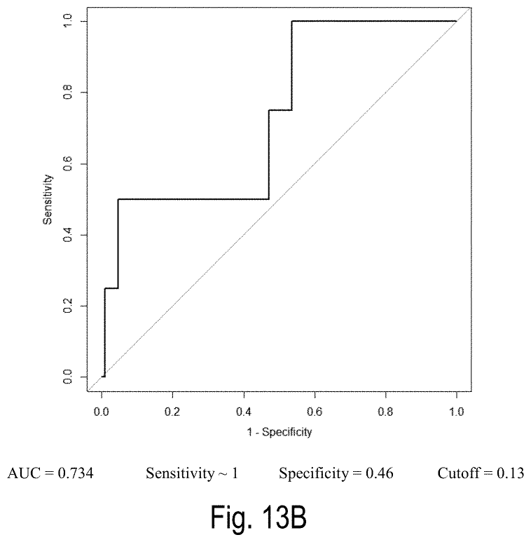

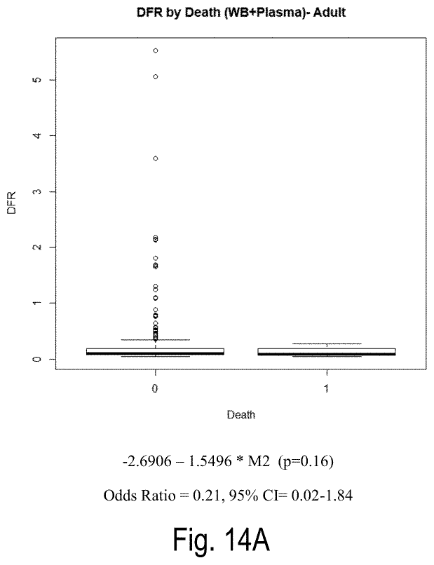

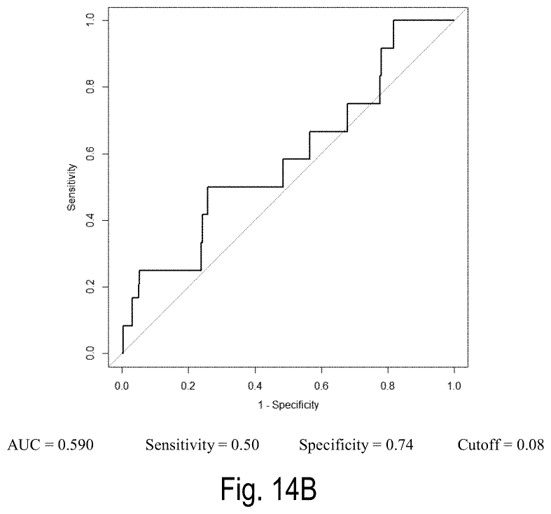

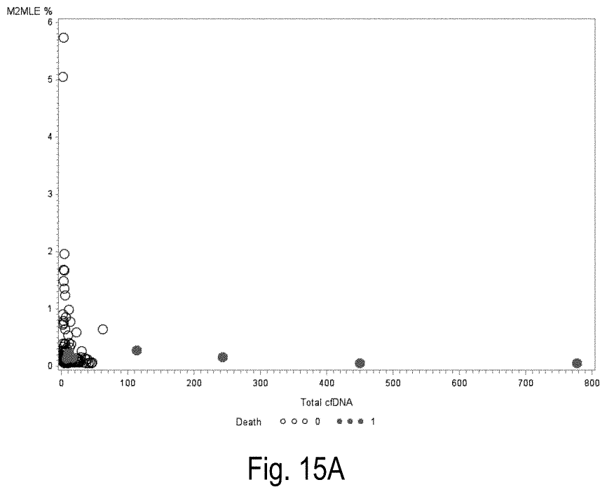

The data was analyzed to examine the donor fraction cell-free DNA (cf-DNA) levels in different samples of subjects who were healthy (e.g., had none of the comorbidities listed above) and those that died. Of the subjects who died, healthy samples not related to death (i.e., samples drawn more than 30 days before death) were included in the healthy group. The “not healthy” group excluded samples from those who did not die as well as samples obtained within 7 days post-transplant. The data is shown in FIGS. 11A-11D (all subjects), 12A-12C (the last sample from each subject), FIGS. 13A-13B (pediatric subjects), FIGS. 14A-14B (adult subjects), and in Tables 6-9 below.

TABLE 6
Analysis of Plasma and Whole Blood Samples (Death vs. Healthy Group) - All Samples
Analysis Variable: m2_raw M2MLE % %

| | | | | | Lower | Upper | | |
|---|---|---|---|---|---|---|---|---|
| death | N | Mean | Std Dev | Median | Quartile | Quartile | Minimum | Maximum |
| 0 | 652 | 0.34 | 0.76 | 0.12 | 0.09 | 0.27 | 0.04 | 11.11 |
| 1 | 18 | 2.27 | 5.73 | 0.14 | 0.08 | 0.27 | 0.04 | 22.25 |

TABLE 7
Analysis of Plasma and Whole Blood Samples (Death vs.
Healthy Group) - Last Sample per Subject
Analysis Variable: m2_raw M2MLE % %

| | | | | | Lower | Upper | | |
|---|---|---|---|---|---|---|---|---|
| death2 | N | Mean | Std Dev | Median | Quartile | Quartile | Minimum | Maximum |
| 0 | 157 | 0.29 | 0.67 | 0.11 | 0.08 | 0.17 | 0.04 | 5.75 |
| 1 | 8 | 0.16 | 0.08 | 0.15 | 0.09 | 0.23 | 0.04 | 0.27 |

TABLE 8
Analysis of Plasma and Whole Blood Samples (Death vs.
Healthy Group) - Pediatric Subjects
Analysis Variable: m2_raw M2MLE % %

| | | | | | Lower | Upper | | |
|---|---|---|---|---|---|---|---|---|
| death | N | Mean | Std Dev | Median | Quartile | Quartile | Minimum | Maximum |
| 0 | 388 | 0.38 | 0.86 | 0.15 | 0.09 | 0.33 | 0.04 | 11.11 |
| 1 | 6 | 6.56 | 8.87 | 2.45 | 0.16 | 11.93 | 0.13 | 22.25 |

TABLE 9
Analysis of Plasma and Whole Blood Samples (Death vs.
Healthy Group) - Adult Subjects
Analysis Variable: m2_raw M2MLE % %

| | | | | | Lower | Upper | | |
|---|---|---|---|---|---|---|---|---|
| death2 | N | Mean | Std Dev | Median | Quartile | Quartile | Minimum | Maximum |
| 0 | 264 | 0.27 | 0.59 | 0.10 | 0.08 | 0.19 | 0.04 | 5.53 |
| 1 | 12 | 0.12 | 0.08 | 0.09 | 0.07 | 0.19 | 0.04 | 0.27 |

Donor Fraction Cell-free DNA (cf-DNA) and Total Cf-DNA

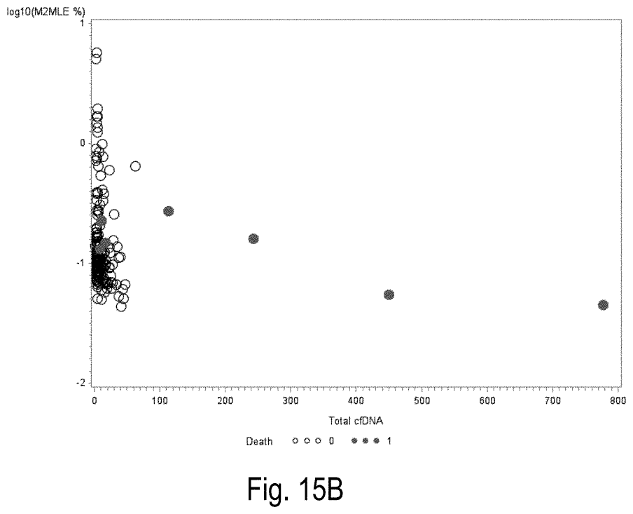

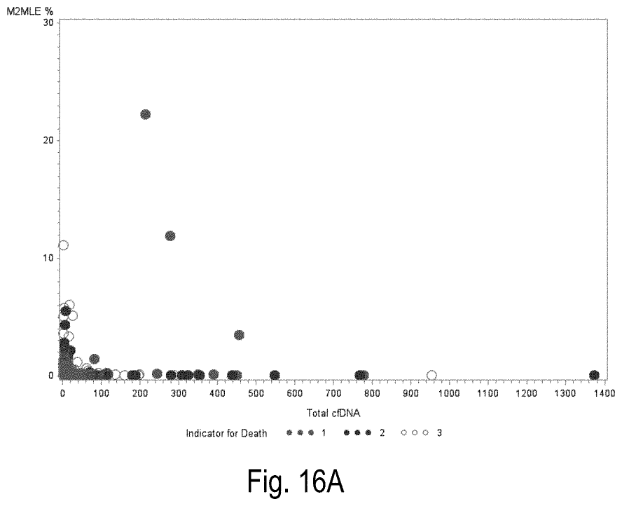

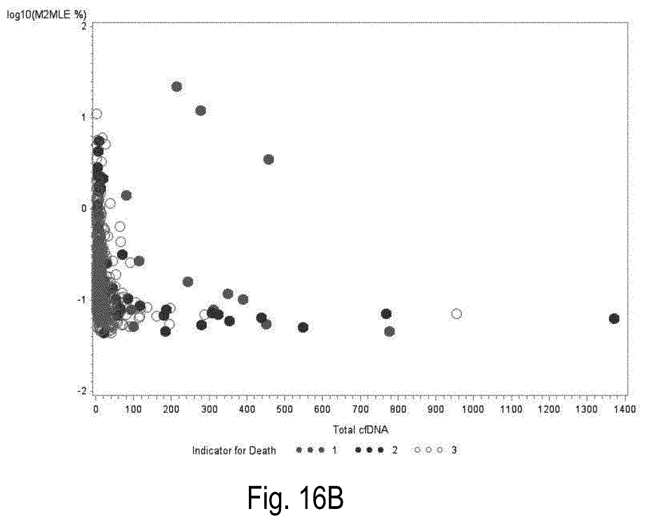

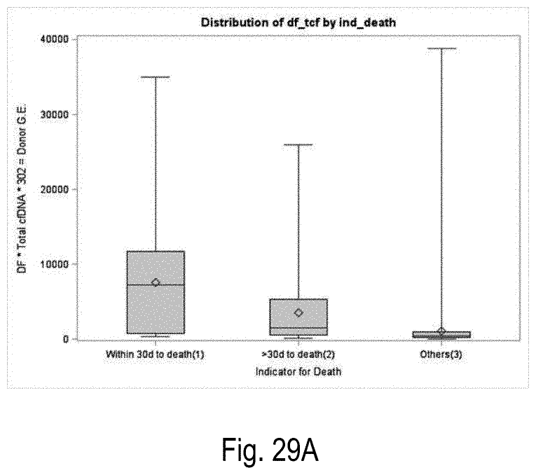

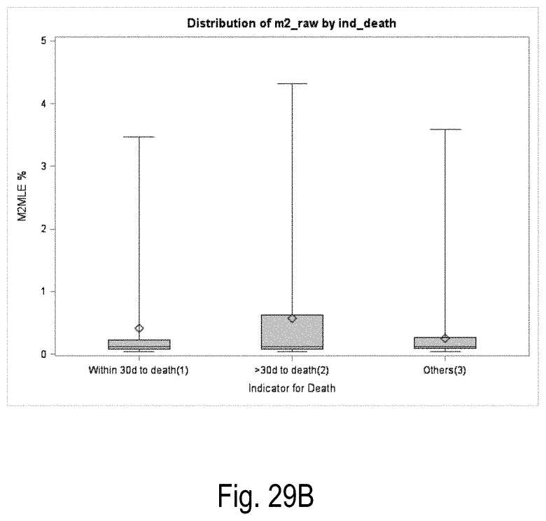

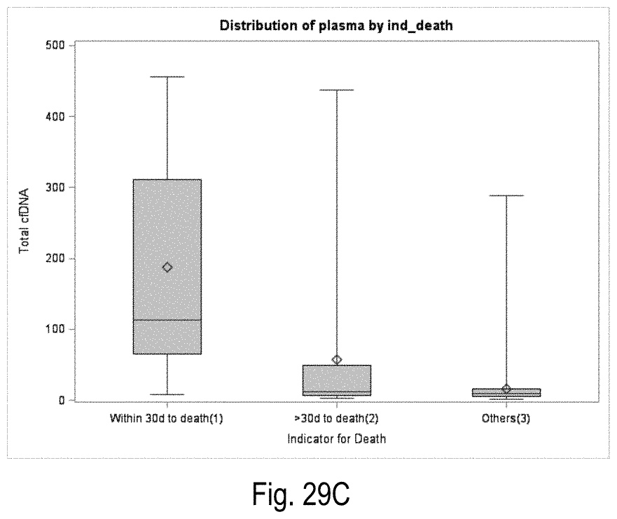

Next, donor fraction cell-free DNA (cf-DNA) was plotted against total cf-DNA, to examine the correlation with death. The results, relating to the last sample per subject, are shown in FIGS. 15A-15B. The data for all samples is shown in FIGS. 16A-16B. Further, the results were analyzed with respect to sample collection time in FIG. 29A. As shown in the boxplot, samples from subjects who died within 30 days of sampling (15 samples) had higher donor fraction cf-DNA and total cf-DNA levels than samples from subjects who died more than 30 days after the samples were obtained (59 samples), and those who did not die during the study (n=593). The data is also presented in Table 10 below. The p-values were as follows: 1 (within 30 days to death) vs. 2 (>30 days to death), p=0.20; 1 vs. 3 (others), p=0.008; and 2 vs. 3, model does not converge. Note that, in the figure, three outliers (DFR*TCF*302>100000) were excluded from the analysis. For comparison purposes, boxplots of donor fraction cf-DNA (FIG. 29B, Table 11) and total cf-DNA (FIG. 29C, Table 12) are also provided. With respect to the donor fraction cf-DNA analysis, the p-values were as follows: 1 vs. 2, p=0.73; 1 vs. 3, model does not converge; and 2 vs. 3, p=0.34. Eight outliers (donor fraction cf-DNA>5) were excluded from the figure. For the total cf-DNA analysis, the p-values were as follows: 1 vs. 2, p=0.13; 1 vs. 3, p=0.04; and 2 vs. 3, model does not converge. Five outliers (total cf-DNA >500) were excluded from the figure. The p-value and the area under the curve increased under the donor fraction cf-DNA×cf-DNA analysis (FIG. 29A) compared to the results obtained with total cf-DNA (FIG. 29C), indicating that the donor fraction cf-DNA and total cf-DNA calculation improves the assay's ability to predict outcome.

TABLE 10
Analysis of Donor Fraction CF-DNA Multiplied by Total CF-DNA as Indicator for Death
Analysis Variable: df_tcf = DF * Total cfDNA * 302 = Donor G.E.

| Indicater for Death | N | Mean | Std Dev | Median | Q1 | Q3 | Minimum | Maximum |
|---|---|---|---|---|---|---|---|---|
| Within 30 d to death (1) | 18 | 168266.95 | 404963.61 | 8335.84 | 1510.13 | 12235.28 | 338.09 | 1439312.93 |
| >30 d to death (2) | 59 | 3556.92 | 4671.94 | 1545.95 | 583.20 | 5291.74 | 127.94 | 25939.82 |
| Others (3) | 593 | 1032.40 | 2591.68 | 468.98 | 234.78 | 954.97 | 49.97 | 38864.79 |

TABLE 11
Analysis of Donor Fraction CF-DNA as Indicator for Death
Analysis Variable: df_tcf = DF * Total cfDNA * 302 = Donor G.E.

| Indicator for Death | N | Mean | Std Dev | Median | Q1 | Q3 | Minimum | Maximum |
|---|---|---|---|---|---|---|---|---|
| Within 30 d to death (1) | 18 | 2.27 | 5.73 | 0.14 | 0.08 | 0.27 | 0.04 | 22.25 |
| >30 d to death (2) | 59 | 0.66 | 1.09 | 0.13 | 0.08 | 0.86 | 0.04 | 5.53 |
| Others (3) | 593 | 0.30 | 0.71 | 0.12 | 0.09 | 0.27 | 0.04 | 11.11 |

TABLE 12
Analysis of Total CF-DNA as Indicator for Death
Analysis Variable: plasma = Total cfDNA

| Indicator for Death | N | Mean | Std Dev | Median | Q1 | Q3 | Minimum | Maximum |
|---|---|---|---|---|---|---|---|---|
| Within 30 d to death(1) | 18 | 220.51 | 207.99 | 163.97 | 65.03 | 349.26 | 8.55 | 777.08 |
| >30 d to death(2) | 59 | 99.77 | 224.65 | 12.97 | 7.19 | 63.12 | 2.17 | 1372.10 |
| Others(3) | 593 | 17.67 | 45.21 | 9.32 | 5.02 | 16.53 | 1.92 | 954.17 |

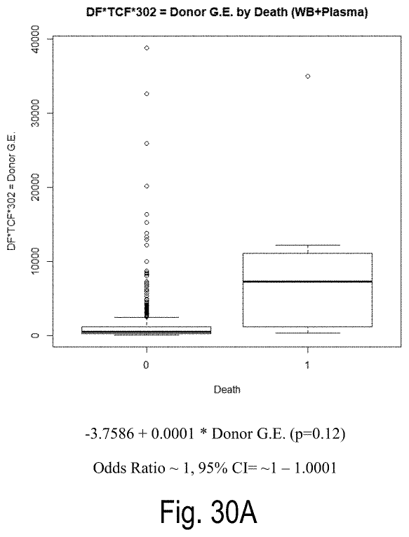

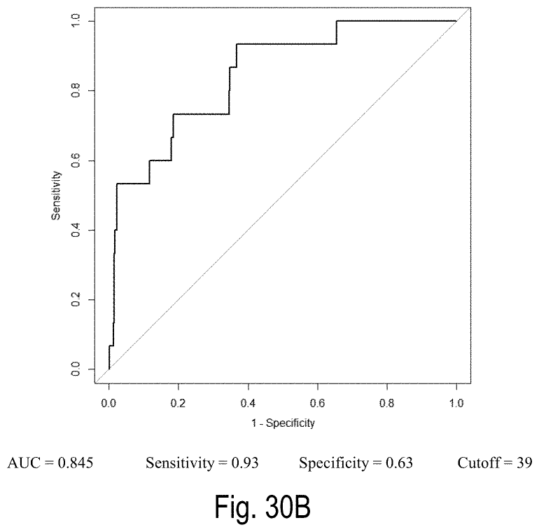

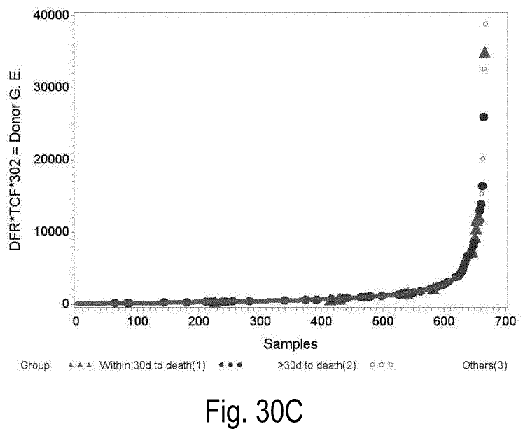

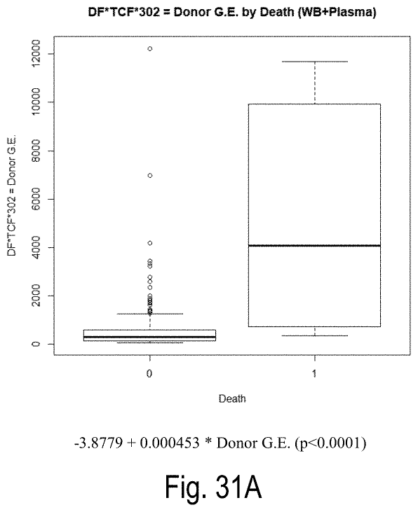

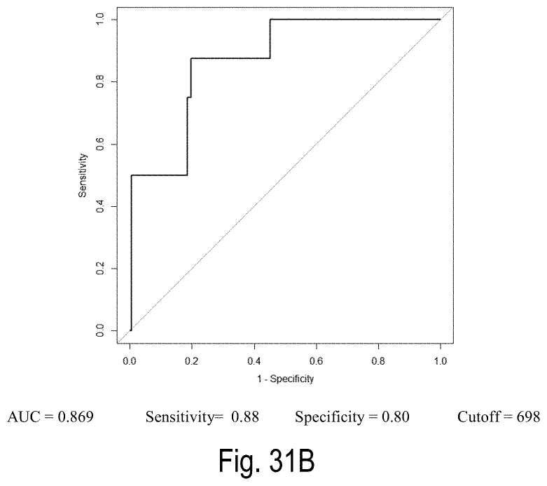

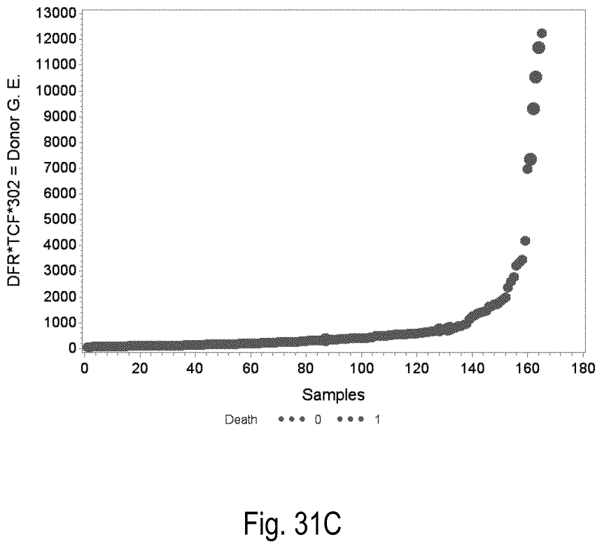

The analysis was repeated with the samples grouped as death or healthy. The healthy cohort included healthy samples not related to death (i.e., those samples drawn more than 30 days before death; Group 2 in the analysis above). The results from all the available samples are presented in FIGS. 30A-30C and in Tables 13 and 14 below. The results from the last sample per subject are presented in FIGS. 31A-31C and Table 15 below.

TABLE 13
Analysis of Donor Fraction CF-DNA Multiplied by Total CF-DNA as Indicator for Death
Analysis Variable: df_tcf = DF * Total cfDNA * 302 = Donor G.E.

| | | | | | Lower | Upper | | |
|---|---|---|---|---|---|---|---|---|
| death | N | Mean | Std Dev | Median | Quartile | Quartile | Minimum | Maximum |
| 0 | 652 | 1260.84 | 2928.83 | 496.91 | 250.89 | 1142.89 | 49.97 | 38864.79 |
| 1 | 18 | 168266.95 | 404963.61 | 8335.84 | 1510.13 | 12235.28 | 338.09 | 1439312.93 |

TABLE 14
Analysis of Donor Fraction CF-DNA Multiplied by Total CF-DNA
as Indicator for Death (excluding 3 outliers from death group)
Analysis Variable: df_tcf = DF * Total cfDNA * 302 = Donor G.E.

| | | | | | Lower | Upper | | |
|---|---|---|---|---|---|---|---|---|
| death | N | Mean | Std Dev | Median | Quartile | Quartile | Minimum | Maximum |
| 0 | 652 | 1260.84 | 2928.83 | 496.91 | 250.89 | 1142.89 | 49.97 | 38864.79 |
| 1 | 15 | 7553.50 | 8932.05 | 7274.74 | 778.55 | 11687.02 | 338.09 | 34996.51 |

TABLE 15
Analysis of Donor Fraction CF-DNA Multiplied by Total CF-DNA
as Indicator for Death (Last Sample per Subject)
Analysis Variable: df_tcf = DF * Total cfDNA * 302 = Donor G.E.

| | | | | | Lower | Upper | | |
|---|---|---|---|---|---|---|---|---|
| death | N | Mean | Std Dev | Median | Quartile | Quartile | Minimum | Maximum |
| 0 | 157 | 658.97 | 1276.31 | 288.23 | 142.60 | 588.95 | 49.97 | 12232.80 |
| 1 | 8 | 5183.64 | 5004.65 | 4067.74 | 728.37 | 9925.90 | 338.09 | 11687.02 |

### Graft Vasculopathy

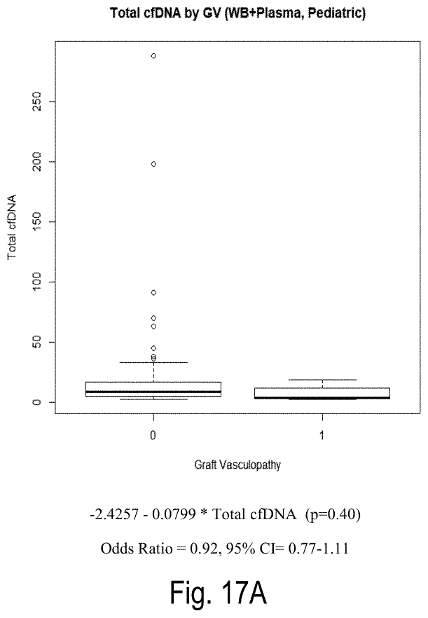

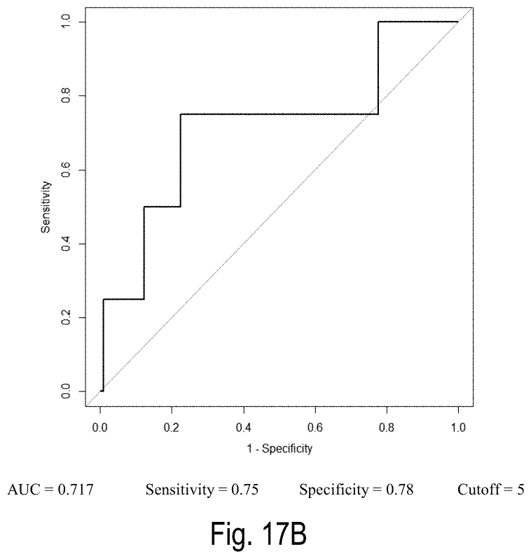

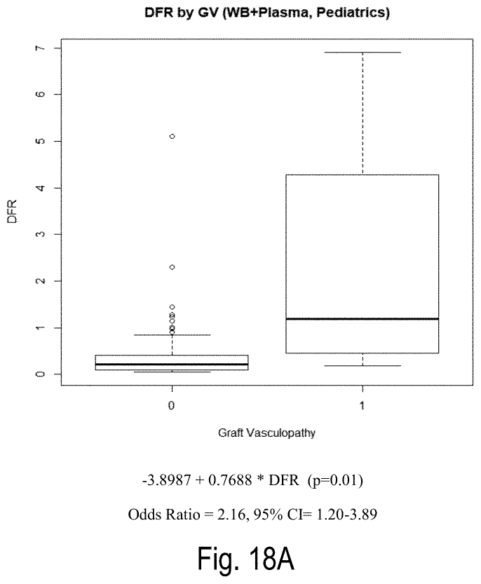

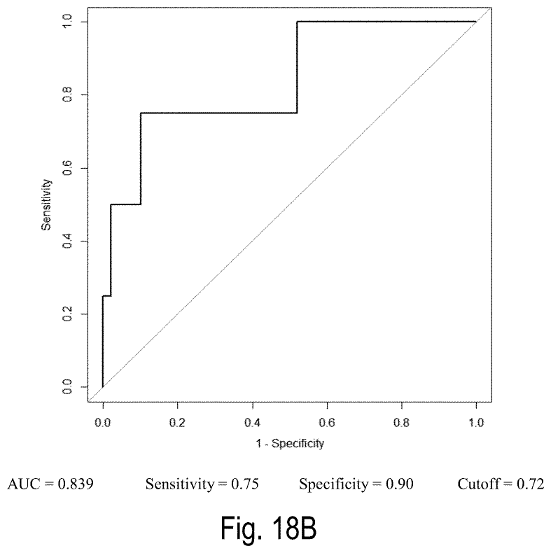

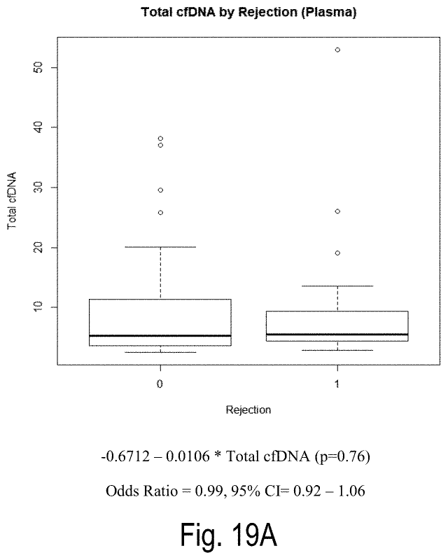

A comparison between samples from subjects with graft vasculopathy compared to healthy controls was undertaken to experimentally determine cutpoints (thresholds). The results are shown in FIGS. 17A-17B (total cf-DNA in pediatric subjects, one sample per subject), FIGS. 18A-18B (donor fraction cf-DNA in pediatric subjects, one sample per subject), and Tables 16-17.

TABLE 16
Analysis of Plasma and Whole Blood Samples (Graft Vasculopathy vs.
Healthy Group) - Pediatric Subjects, 1 sample/subject (total cf-DNA)
Analysis Variable: plasma Total cfDNA

| Graft | N | | | | Lower | Upper | | |
|---|---|---|---|---|---|---|---|---|
| Vasculopathy | Obs | Mean | Std Dev | Median | Quartile | Quartile | Minimum | Maximum |
| 0 | 98 | 17.94 | 36.18 | 8.80 | 4.88 | 16.80 | 2.45 | 288.19 |
| 1 | 4 | 7.46 | 7.79 | 4.16 | 3.03 | 11.88 | 2.45 | 19.05 |

TABLE 17
Analysis of Plasma and Whole Blood Samples (Graft Vasculopathy vs.
Healthy Group) - Pediatric Subjects, 1 sample/subject (donor fraction cf-DNA)
Analysis Variable: m2_raw M2MLE %%

| Graft | | | | | Lower | Upper | | |
|---|---|---|---|---|---|---|---|---|
| Vasculopathy | N | Mean | Std Dev | Median | Quartile | Quartile | Minimum | Maximum |
| 0 | 98 | 0.37 | 0.60 | 0.21 | 0.09 | 0.41 | 0.04 | 5.11 |
| 1 | 4 | 2.37 | 3.09 | 1.19 | 0.45 | 4.29 | 0.18 | 6.91 |

Rejection (Acute Cellular Rejection and/or Antibody-mediated Rejection)

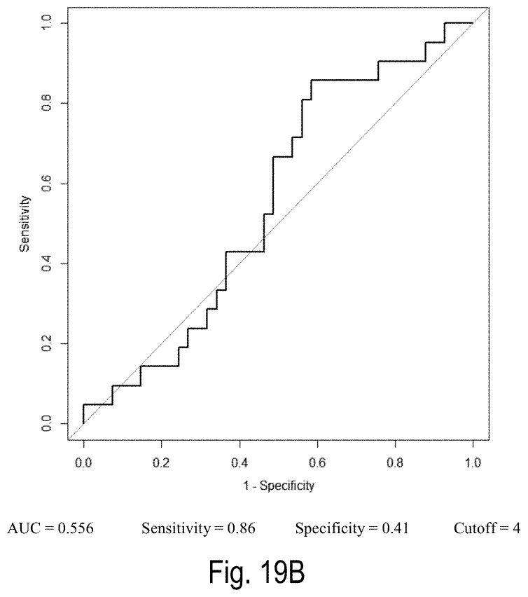

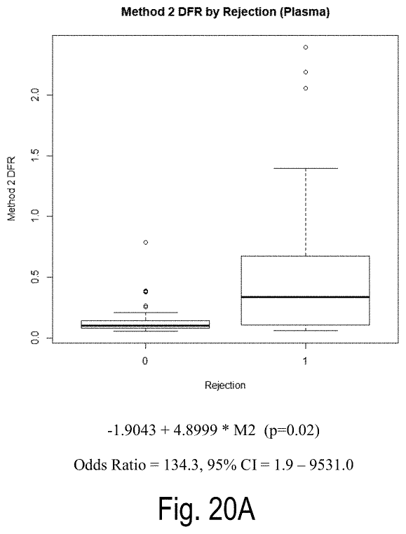

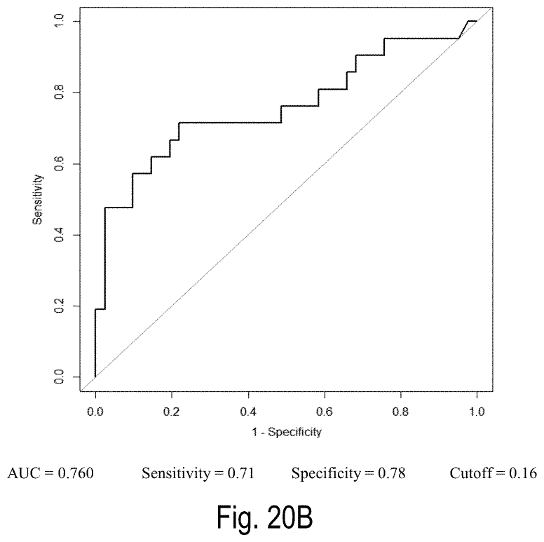

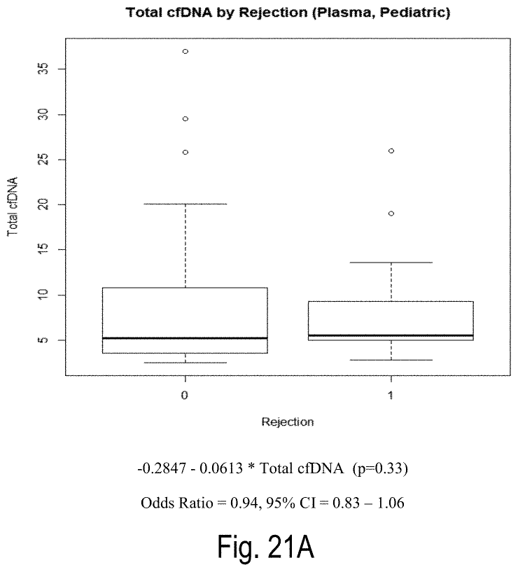

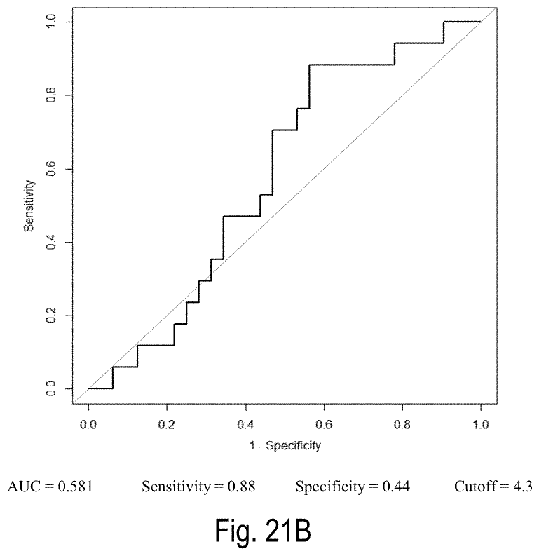

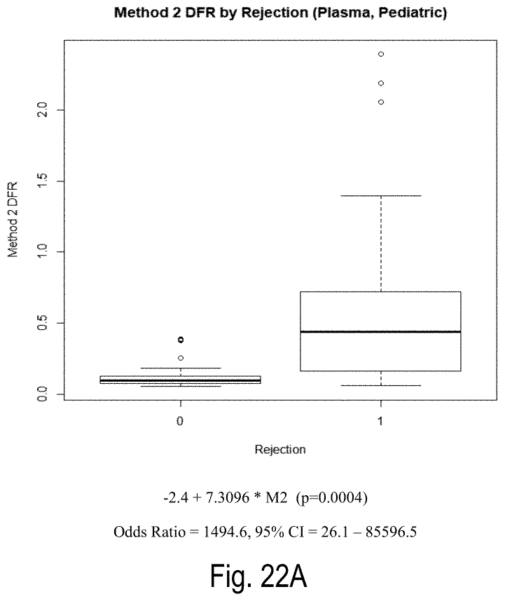

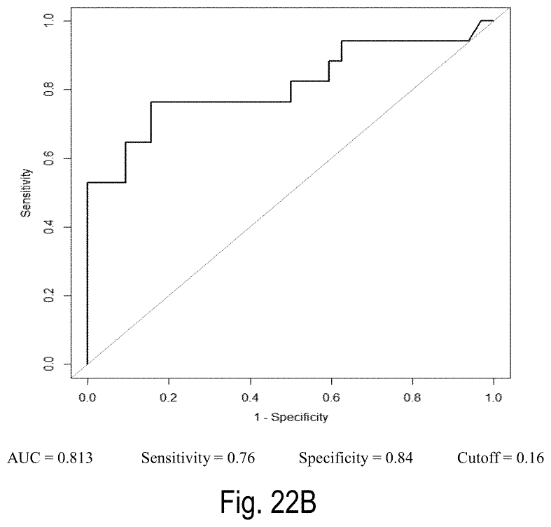

A comparison between samples from subjects with rejection compared to healthy controls was undertaken to experimentally determine cutpoints (thresholds). Rejection, in this instance, referred to acute cellular rejection (ACR) levels of 1, 2, or 3 and/or antibody-mediated rejection (AMR) levels of 1 or 2. The results are shown in FIGS. 19A-19B (total cf-DNA in plasma samples), FIGS. 20A-20B (donor fraction cf-DNA in plasma samples), FIGS. 21A-21B (total cf-DNA in plasma samples from pediatric subjects), FIGS. 22A-22B (donor fraction cf-DNA in plasma samples from pediatric subjects), and Tables 18-21.

TABLE 18
Analysis of Plasma Samples (Rejection vs. Healthy Group) - Total cf-DNA
Analysis Variable: plasma Total cfDNA

| ACR1/2/3 and/ | | | | | Lower | Upper | | |
|---|---|---|---|---|---|---|---|---|
| or AMR1/2 | N | Mean | Std Dev | Median | Quartile | Quartile | Minimum | Maximum |
| 0 | 41 | 9.37 | 9.16 | 5.22 | 3.56 | 11.30 | 2.47 | 38.16 |
| 1 | 21 | 9.99 | 11.37 | 5.52 | 4.42 | 9.31 | 2.78 | 53.03 |

TABLE 19
Analysis of Plasma Samples (Rejection vs.
Healthy Group) - Donor fraction cf-DNA (MOMA)
Analysis Variable: m2_raw M2MLE %%

| ACR1/2/3 and/ | | | | | Lower | Upper | | |
|---|---|---|---|---|---|---|---|---|
| or AMR1/2 | N | Mean | Std Dev | Median | Quartile | Quartile | Minimum | Maximum |
| 0 | 41 | 0.15 | 0.13 | 0.10 | 0.08 | 0.14 | 0.05 | 0.79 |
| 1 | 21 | 0.63 | 0.74 | 0.34 | 0.11 | 0.67 | 0.06 | 2.40 |

TABLE 20
Analysis of Plasma Samples (Rejection vs.
Healthy Group) - Total cf-DNA (Pediatric Samples)
Analysis Variable: plasma Total cfDNA

| ACR1/2/3 and/ | | | | | Lower | Upper | | |
|---|---|---|---|---|---|---|---|---|
| or AMR1/2 | N | Mean | Std Dev | Median | Quartile | Quartile | Minimum | Maximum |
| 0 | 32 | 8.84 | 8.50 | 5.20 | 3.56 | 10.78 | 2.47 | 37.02 |
| 1 | 17 | 8.48 | 6.08 | 5.52 | 4.98 | 9.31 | 2.78 | 26.00 |

TABLE 21
Analysis of Plasma Samples (Rejection vs.
Healthy Group) - Donor fraction cf-DNA (MOMA) of Pediatric Samples
Analysis Variable: m2_raw M2MLE %%

| ACR 1/2/3 and/ | | | | | Lower | Upper | | |
|---|---|---|---|---|---|---|---|---|
| or AMR1/2 | N | Mean | Std Dev | Median | Quartile | Quartile | Minimum | Maximum |
| 0 | 32 | 0.13 | 0.09 | 0.10 | 0.08 | 0.13 | 0.05 | 0.39 |
| 1 | 17 | 0.73 | 0.79 | 0.44 | 0.16 | 0.72 | 0.06 | 2.40 |

### Treatment for Infection

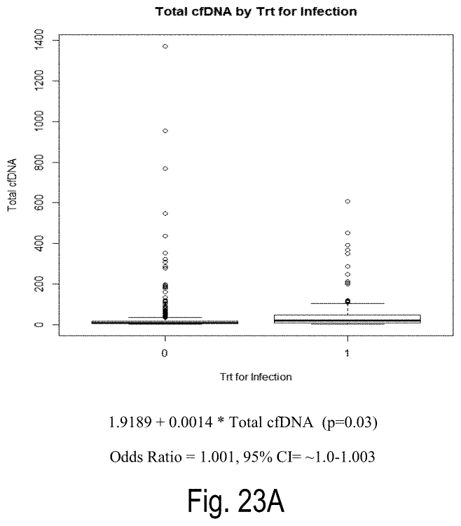

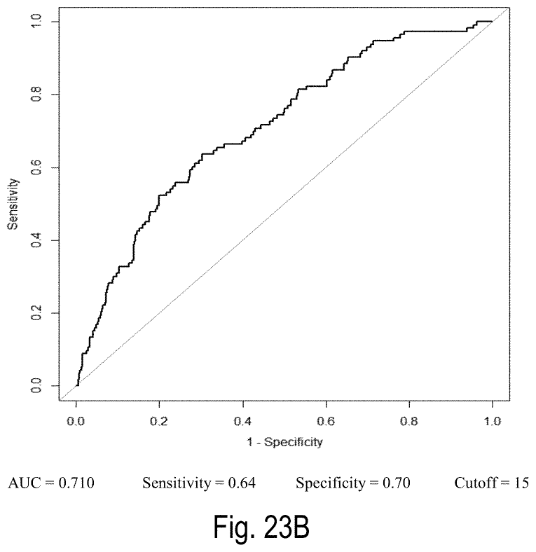

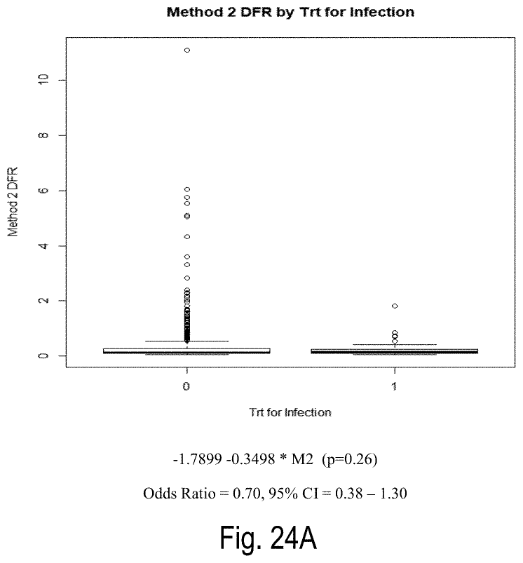

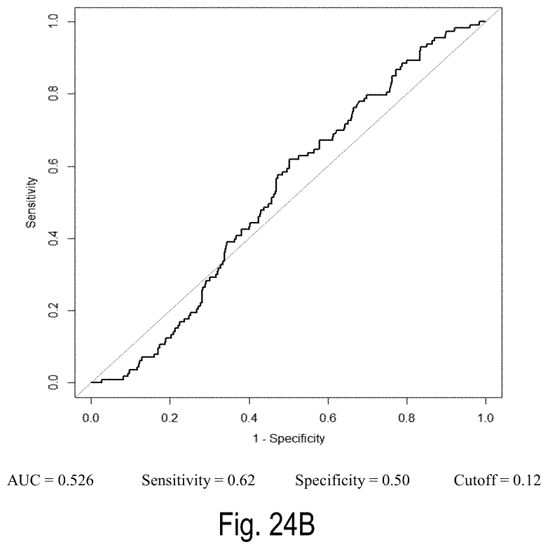

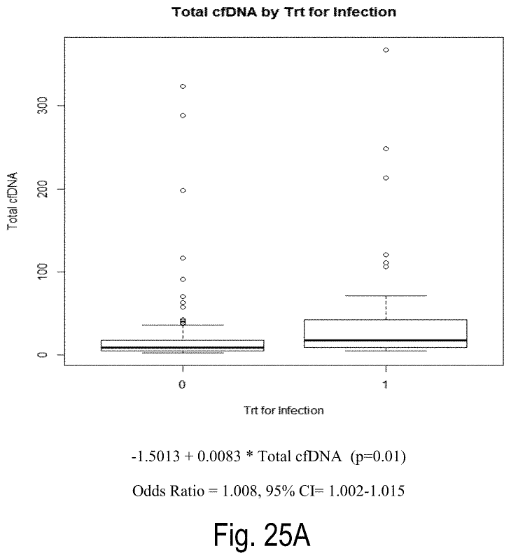

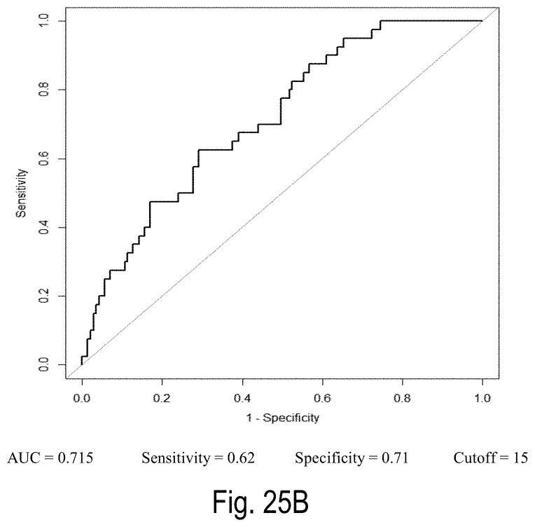

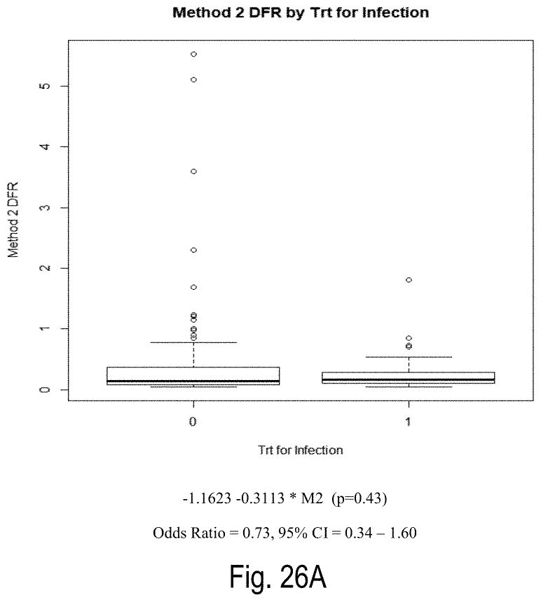

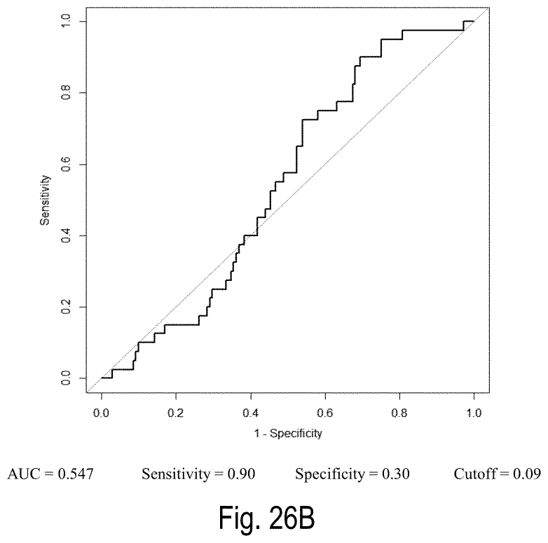

A comparison between samples from subjects with infection (e.g., those treated for infection) compared to healthy controls was undertaken to experimentally determine cutpoints (thresholds). The results are shown in FIGS. 23A-23B (total cf-DNA in plasma and whole blood samples), FIGS. 24A-24B (donor fraction cf-DNA in plasma and whole blood samples), FIGS. 25A-25B (total cf-DNA in plasma and whole blood samples; one sample per subject), FIGS. 26A-26B (donor fraction cf-DNA in plasma and whole blood samples; one sample per subject), and Tables 22-25

TABLE 22
Analysis of Plasma and Whole Blood Samples (Infection vs.
Healthy Group) - Total cf-DNA
Analysis Variable: plasma Total cfDNA

| | | | | | Lower | Upper | | |
|---|---|---|---|---|---|---|---|---|
| infection | N | Mean | Std Dev | Median | Quartile | Quartile | Minimum | Maximum |
| 0 | 652 | 25.10 | 83.13 | 9.89 | 5.09 | 17.52 | 1.92 | 1372.10 |
| 1 | 113 | 55.26 | 97.54 | 21.74 | 9.93 | 48.87 | 2.81 | 608.19 |

TABLE 23
Analysis of Plasma and Whole Blood Samples (Infection vs.
Healthy Group) - Donor Fraction cf-DNA (MOMA)
Analysis Variable: m2_raw M2MLE %%

| Treatment | | | | | Lower | Upper | | |
|---|---|---|---|---|---|---|---|---|
| for Infection | N | Mean | Std Dev | Median | Quartile | Quartile | Minimum | Maximum |
| 0 | 652 | 0.34 | 0.76 | 0.12 | 0.09 | 0.27 | 0.04 | 11.11 |
| 1 | 113 | 0.21 | 0.21 | 0.14 | 0.10 | 0.25 | 0.05 | 1.81 |

TABLE 24
Analysis of Plasma and Whole Blood Samples (Infection vs.
Healthy Group) - Total cf-DNA (one sample per subject)
Analysis Variable: plasma Total cfDNA

| | | | | | Lower | Upper | | |
|---|---|---|---|---|---|---|---|---|
| infection | N | Mean | Std Dev | Median | Quartile | Quartile | Minimum | Maximum |
| 0 | 141 | 19.43 | 41.08 | 9.04 | 4.77 | 17.66 | 2.32 | 323.61 |
| 1 | 40 | 46.60 | 74.60 | 17.31 | 9.28 | 42.42 | 4.78 | 367.59 |

TABLE 25
Analysis of Plasma and Whole Blood Samples (Infection vs.
Healthy Group) - Donor Fraction cf-DNA (MOMA); one sample per subject
Analysis Variable: m2_raw M2MLE %%

| Treatment | | | | | Lower | Upper | | |
|---|---|---|---|---|---|---|---|---|
| for Infection | N | Mean | Std Dev | Median | Quartile | Quartile | Minimum | Maximum |
| 0 | 141 | 0.37 | 0.73 | 0.15 | 0.09 | 0.37 | 0.04 | 5.53 |
| 1 | 40 | 0.27 | 0.31 | 0.17 | 0.11 | 0.28 | 0.05 | 1.81 |

### Risk Stratification

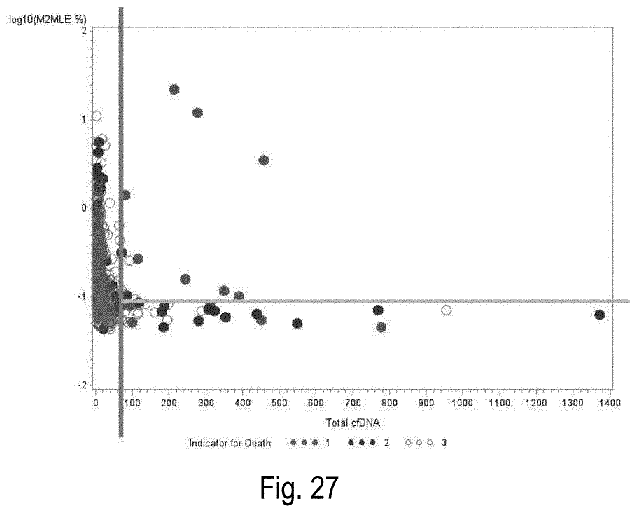

Two different methods of stratifying risk were explored. The first, classification of regression trees (CART; FIG. 27) showed high sensitivity and specificity. As shown in the Figure, when levels are left of the vertical line, the subjects have a low chance of death. To the right of the vertical line, the subjects with values above the horizontal line have a high chance (risk) of death, while the subjects with values below the horizontal line are at medium risk.

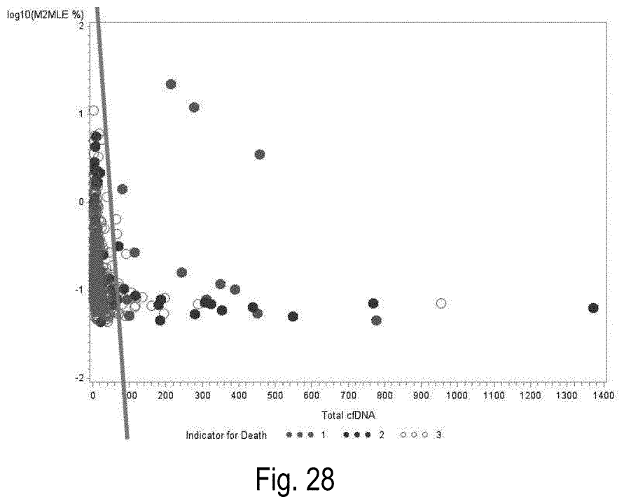

In the second method, linear discriminant analysis (LDA; FIG. 28), the most separating diagonal line is determined. The distance to the diagonal line is added to the ROC analysis to further determine a given subject's risk of death.

TABLE 26
Utility of Genome Equivalent (GE): Death vs. Healthy
Group Using Last Sample per Subject

| | N | Mean | Std Dev | Range |
|---|---|---|---|---|
| Healthy | 157 | 658.97 | ±1276.3 | 49.9-12232.8 |
| Death | 8 | 5183.64 | ±5004.6 | 338.1-11687.1 |

Analysis Variable: df_tcf = DF * Tota cfDNA * 302 = Donor GE

1. A method of assessing a sample from a transplant subject, the method comprising:
(a) determining the product of an amount of donor-specific cell-free DNA (DS cf-DNA) and an amount of total cf-DNA in the sample; and
(b) reporting and/or recording the product.

2. The method of claim 1, wherein one or more further products of amounts of DS cf-DNA and total cf-DNA are determined each from a sample taken from the subject at a different point in time.

3. The method of claim 1, wherein the one or more further products are determined from samples taken from the subject monthly or bimonthly.

4. (canceled)

5. The method of claim 1, wherein the method further comprises:
(c) comparing the product(s) to threshold value(s) and/or product value(s) from one or more prior time points.

6. The method of claim 5, wherein the method further comprises:
(d) determining and/or assigning a risk to the subject based on a comparison of the product(s) to threshold value(s) and/or product value(s) from one or more prior time points.

7. A method of treating or monitoring a transplant subject, the method comprising:
(a) obtaining an amount of donor-specific cell-free DNA (DS cf-DNA) and an amount of total cf-DNA, or product thereof, in a sample from the subject; and
(b) comparing a/the product of the amount of DS cf-DNA and total cf-DNA to a threshold value or product value from a prior time point; and
(c) determining a treatment or monitoring regimen for the subject based on the comparison.

8. The method of claim 7, further comprising obtaining an amount of donor-specific cell-free DNA (DS cf-DNA) and an amount of total cf-DNA, or product thereof, in a sample from the subject from another time point.

9. The method of claim 7, wherein the an amount of DS cf-DNA and total cf-DNA, or product thereof, are/is obtained from samples taken from the subject monthly or bimonthly.

10.-12. (canceled)

13. The method of claim 1, wherein the amounts and/or values are provided in a report or are recorded in a database.

14.-16 (canceled)

17. The method of claim 1, wherein a product value that is greater than a threshold value and/or product value from a different time point represents an increased or increasing risk.

18. The method of claim 1, wherein a product value that is lower than a threshold value and/or product value from a different time point represents a decreased or decreasing risk.

19. The method of claim 1, wherein the determining a monitoring regimen comprises determining the amount of DS cf-DNA and total cf-DNA, or product thereof, in the subject over time or at a subsequent point in time, or suggesting such monitoring to the subject.

20. The method of claim 1, wherein the time between samples is decreased if the product value is increased relative to threshold(s) or product value(s) from different time point(s).

21. The method of claim 1, wherein the time between samples is increased if the product value is decreased relative to threshold(s) or product value(s) from different time point(s).

22. The method of claim 1, wherein the determining a monitoring regimen comprises using or suggesting the use of one or more additional test(s) to assess the subject.

23. The method of claim 1, wherein the determining a treatment regimen comprises selecting or suggesting a treatment for the subject, or changing the treatment of the subject, or suggesting such change.

24. The method of claim 1, wherein the determining a treatment regimen comprises treating the subject.

25. The method of claim 1, wherein the determining a treatment regimen comprises providing information about a treatment to the subject.

26. The method of claim 1, wherein the sample is a blood, plasma or serum sample.

27.-28. (canceled)

29. The method of claim 1, wherein the transplant subject is a heart transplant subject.

30.-32. (canceled)
